SEQUENCE LISTING (12 sequences)

SEQ ID NO: 1 - DNA, 3677 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 2 - DNA, 738 bp, Homo sapiens
agtctcagattggtaaacacccgaactggtcaactctcaagagaccatctggttcaggttcctgactgggccagcgagtgaggatggccaggcctctggagcaggcggtagctgccatcgtgtgcaccttccaggaatacgcagggcgctgtggggacaaatacaagctctgccaggcggagctcaaggagctgctgcagaaggagctggccacctggaccccgactgagtttcgggaatgtgactacaacaaattcatgagtgttctggacaccaacaaggactgcgaggtggactttgtggagtatgtgcgctcacttgcctgcctctgtctctactgccacgagtacttcaaggactgcccctcagagcccccctgctcccagtagcctctgctccagggggtgcgctggctgtcgggggctgggcatgtctcccacaccccctcctaccctctctcctgtacccctttcaatctggacttgcccaggtcttctgcgatcagttaacccattttacctaggaggcccagagatgtgagggctccttcctcaggatgcccagcgaatgaggggtagagccactctggggcccagcctgcctgccgcacccctgtggcctcccttgtggatgggaggaggcgggatctgctctgaggccctcgaggctcagcagagcgtgcaccaatgagaccacgatgggaaagggcctatttaactcctaataaaaaactggcat

SEQ ID NO: 3 - DNA, 4087 bp, Homo sapiens
tctcccgagaacctcaccccccggtattcccctcaccctgaagtaagggagtctccctcttcccctcagctcctaaccaaggtgctcagtaccgcggctagggtgcccagcgggaggctgggtagttaccggaggaggcagagccggtcgcacgcctgggtcccagcggaggcccagtcgactctcgacccgggccccagcctccttgcgtggcgaaggtcgtgcctcaggtctcctggcgaacctggccgcccggaagcggagagtgactcctccccttcgtcccctcggtgcccgccaggcggcgccgtcggctgggcccggattcccctgcggcttcgatccctttccactgggtattgagattggcgaggggagaggcgcagaaacggcgtgatgcagaaagcctcagtgttgctcttcctggcctgggtctgcttcctcttctacgctggcattgccctcttcaccagtggcttcctgctcacccgtttggagctcaccaaccatagcagctgccaagagcccccaggccctgggtccctgccatgggggagccaagggaaacctggggcctgctggatggcttcccgattttcgcgggttgtgttggtgctgatagatgctctgcgatttgacttcgcccagccccagcattcacacgtgcctagagagcctcctgtctccctacccttcctgggcaaactaagctccttgcagaggatcctggagattcagccccaccatgcccggctctaccgatctcaggttgaccctcctaccaccaccatgcagcgcctcaaggccctcaccactggctcactgcctacctttattgatgctggtagtaacttcgccagccacgccatagtggaagacaatctcattaagcagctcaccagtgcaggaaggcgtgtagtcttcatgggagatgatacctggaaagaccttttccctggtgctttctccaaagctttcttcttcccatccttcaatgtcagagacctagacacagtggacaatggcatcctggaacacctctaccccaccatggacagtggtgaatgggacgtgctgattgctcacttcctgggtgtggaccactgtggccacaagcatggccctcaccaccctgaaatggccaagaaacttagccagatggaccaggtgatccagggacttgtggagcgtctggagaatgacacactgctggtagtggctggggaccatgggatgaccacaaatggagaccatggaggggacagtgagctggaggtctcagctgctctctttctgtatagccccacagcagtcttccccagcaccccaccagaggagccagaggtgattcctcaagttagccttgtgcccacgctggccctgctgctgggcctgcccatcccatttgggaatatcggggaagtgatggctgagctattctcagggggtgaggactcccagccccactcctctgctttagcccaagcctcagctctccatctcaatgctcagcaggtgtcccgatttcttcatacctactcagctgctactcaggaccttcaagctaaggagcttcatcagctgcagaacctcttctccaaggcctctgctgactaccagtggcttctccagagccccaagggggctgaggcgacactgccgactgtgattgctgagctgcagcagttcctgcggggagctcgggccatgtgcatcgagtcttgggctcgtttctctctggtccgcatggcggggggtactgctctcttggctgcttcctgctttatctgcctgctggcatctcagtgggcaatatccccaggctttccattctgccctctactcctgacacctgtggcctggggcctggttggggccatagcgtatgctggactcctgggaactattgagctgaagctagatctagtgcttctaggggctgtggctgcagtgagctcattcctcccttttctgtggaaagcctgggctggctgggggtccaagaggcccctggcaaccctgtttcccatccctgggcccgtcctgttactcctgctgtttcgcttggctgtgttcttctctgatagttttgttgtagctgaggccagggccacccccttccttttgggctcattcatcctgctcctggttgtccagcttcactgggagggccagctgcttccacctaagctactcacaatgccccgccttggcacttcagccacaacaaaccccccacggcacaatggtgcatatgccctgaggcttggaattgggttgcttttatgtacaaggctagctgggctttttcatcgttgccctgaagagacacctgtttgccactcctctccctggctgagtcctctggcatccatggtgggtggtcgagccaagaatttgtggtatggagcttgtgtggcggcgctggtggccctgttagctgccgtgcgcttgtggcttcgccgctatggtaatctcaagagccccgagccacccatgctctttgtgcgctggggactgcccctaatggcattgggtactgctgcctactgggcattggcgtcgggggcagatgaggctcccccccgtctccgggtcctggtctctggggcatccatggtgctgcctcgggctgtagcagggctggctgcttcagggctcgcgctgctgctctggaagcctgtgacagtgctggtgaaggctggggcaggcgctccaaggaccaggactgtcctcactcccttctcaggcccccccacttctcaagctgacttggattatgtggtccctcaaatctaccgacacatgcaggaggagttccggggccggttagagaggaccaaatctcagggtcccctgactgtggctgcttatcagttggggagtgtctactcagctgctatggtcacagccctcaccctgttggccttcccacttctgctgttgcatgcggagcgcatcagccttgtgttcctgcttctgtttctgcagagcttccttctcctacatctgcttgctgctgggatacccgtcaccacccctggtccttttactgtgccatggcaggcagtctcggcttgggccctcatggccacacagaccttctactccacaggccaccagcctgtctttccagccatccattggcatgcagccttcgtgggattcccagagggtcatggctcctgtacttggctgcctgctttgctagtgggagccaacacctttgcctcccacctcctctttgcagtaggttgcccactgctcctgctctggcctttcctgtgtgagagtcaagggctgcggaagagacagcagcccccagggaatgaagctgatgccagagtcagacccgaggaggaagaggagccactgatggagatgcggctccgggatgcgcctcagcacttctatgcagcactgctgcagctgggcctcaagtacctctttatccttggtattcagattctggcctgtgccttggcagcctccatccttcgcaggcatctcatggtctggaaagtgtttgcccctaagttcatatttgaggctgtgggcttcattgtgagcagcgtgggacttctcctgggcatagctttggtgatgagagtggatggtgctgtgagctcctggttcaggcagctatttctggcccagcagaggtagcctagtctgtgattactggcacttggctacagagagtgcttgagaacagtgtagcctggcctgtacaggtactggatgatctgcaagacaggctcagccatactcttactatcatgcagccaggggccgctgacatctaggacttcattattctataattcaggaccacagtggagtatgatccctaactcctgatttggatgcatctgagggacaaggggggcggtctccgaagtggaataaaataggccgggcgtggtgacttgcacctataatcccagcactttgggaggcagaggtgggaggattgcttggtcccaggagttcaagaccagcctgtggaacataacaagaccccgtctctactatttaaaaaaaagtgtaataaaatgataatataattaaaaaaaaaaaaaaaaaa

SEQ ID NO: 4 - DNA, 4246 bp, Homo sapiens
gctctcactctggctgggagcagaaggcagcctcggtctctgggcggcggcggcggcccactctgccctggccgcgctgtgtggtgaccgcaggccccagacatgagggcggcccgtgctctgctgcccctgctgctgcaggcctgctggacagccgcgcaggatgagccggagaccccgagggccgtggccttccaggactgccccgtggacctgttctttgtgctggacacctctgagagcgtggccctgaggctgaagccctacggggccctcgtggacaaagtcaagtccttcaccaagcgcttcatcgacaacctgagggacaggtactaccgctgtgaccgaaacctggtgtggaacgcaggcgcgctgcactacagtgacgaggtggagatcatccaaggcctcacgcgcatgcctggcggccgcgacgcactcaaaagcagcgtggacgcggtcaagtactttgggaagggcacctacaccgactgcgctatcaagaaggggctggagcagctcctcgtggggggctcccacctgaaggagaataagtacctgattgtggtgaccgacgggcaccccctggagggctacaaggaaccctgtggggggctggaggatgctgtgaacgaggccaagcacctgggcgtcaaagtcttctcggtggccatcacacccgaccacctggagccgcgtctgagcatcatcgccacggaccacacgtaccggcgcaacttcacggcggctgactggggccagagccgcgacgcagaggaggccatcagccagaccatcgacaccatcgtggacatgatcaaaaataacgtggagcaagtgtgctgctccttcgaatgccagcctgcaagaggacctccggggctccggggcgaccccggctttgagggagaacgaggcaagccggggctcccaggagagaagggagaagccggagatcctggaagacccggggacctcggacctgttgggtaccagggaatgaagggagaaaaagggagccgtggggagaagggctccaggggacccaagggctacaagggagagaagggcaagcgtggcatcgacggggtggacggcgtgaagggggagatggggtacccaggcctgccaggctgcaagggctcgcccgggtttgacggcattcaaggaccccctggccccaagggagaccccggtgcctttggactgaaaggagaaaagggcgagcctggagctgacggggaggcggggagaccagggagctcgggaccatctggagacgagggccagccgggagagcctgggccccccggagagaaaggagaggcgggcgacgaggggaacccaggacctgacggtgcccccggggagcggggtggccctggagagagaggaccacgggggaccccaggcacgcggggaccaagaggagaccctggtgaagctggcccgcagggtgatcagggaagagaaggccccgttggtgtccctggagacccgggcgaggctggccctatcggacctaaaggctaccgaggcgatgagggtcccccagggtccgagggtgccagaggagccccaggacctgccggaccccctggagacccggggctgatgggtgaaaggggagaagacggccccgctggaaatggcaccgagggcttccccggcttccccgggtatccgggcaacaggggcgctcccgggataaacggcacgaagggctaccccggcctcaagggggacgagggagaagccggggaccccggagacgataacaacgacattgcaccccgaggagtcaaaggagcaaaggggtaccggggtcccgagggcccccagggacccccaggacaccaaggaccgcctgggccggacgaatgcgagattttggacatcatcatgaaaatgtgctcttgctgtgaatgcaagtgcggccccatcgacctcctgttcgtgctggacagctcagagagcattggcctgcagaacttcgagattgccaaggacttcgtcgtcaaggtcatcgaccggctgagccgggacgagctggtcaagttcgagccagggcagtcgtacgcgggtgtggtgcagtacagccacagccagatgcaggagcacgtgagcctgcgcagccccagcatccggaacgtgcaggagctcaaggaagccatcaagagcctgcagtggatggcgggcggcaccttcacgggggaggccctgcagtacacgcgggaccagctgctgccgcccagcccgaacaaccgcatcgccctggtcatcactgacgggcgctcagacactcagagggacaccacaccgctcaacgtgctctgcagccccggcatccaggtggtctccgtgggcatcaaagacgtgtttgacttcatcccaggctcagaccagctcaatgtcatttcttgccaaggcctggcaccatcccagggccggcccggcctctcgctggtcaaggagaactatgcagagctgctggaggatgccttcctgaagaatgtcaccgcccagatctgcatagacaagaagtgtccagattacacctgccccatcacgttctcctccccggctgacatcaccatcctgctggacggctccgccagcgtgggcagccacaactttgacaccaccaagcgcttcgccaagcgcctggccgagcgcttcctcacagcgggcaggacggaccccgcccacgacgtgcgggtggcggtggtgcagtacagcggcacgggccagcagcgcccagagcgggcgtcgctgcagttcctgcagaactacacggccctggccagtgccgtcgatgccatggactttatcaacgacgccaccgacgtcaacgatgccctgggctatgtgacccgcttctaccgcgaggcctcgtccggcgctgccaagaagaggctgctgctcttctcagatggcaactcgcagggcgccacgcccgctgccatcgagaaggccgtgcaggaagcccagcgggcaggcatcgagatcttcgtggtggtcgtgggccgccaggtgaatgagccccacatccgcgtcctggtcaccggcaagacggccgagtacgacgtggcctacggcgagagccacctgttccgtgtccccagctaccaggccctgctccgcggtgtcttccaccagacagtctccaggaaggtggcgctgggctagcccaccctgcacgccggcaccaaaccctgtcctcccacccctccccactcatcactaaacagagtaaaatgtgatgcgaattttcccgaccaacctgattcgctagattttttttaaggaaaagcttggaaagccaggacacaacgctgctgcctgctttgtgcagggtcctccggggctcagccctgagttggcatcacctgcgcagggccctctggggctcagccctgagctagtgtcacctgcacagggccctctgaggctcagccctgagctggcgtcacctgtgcagggccctctggggctcagccctgagctggcctcacctgggttccccaccccgggctctcctgccctgccctcctgcccgccctccctcctgcctgcgcagctccttccctaggcacctctgtgctgcatcccaccagcctgagcaagacgccctctcggggcctgtgccgcactagcctccctctcctctgtccccatagctggtttttcccaccaatcctcacctaacagttactttacaattaaactcaaagcaagctcttctcctcagcttggggcagccattggcctctgtctcgttttgggaaaccaaggtcaggaggccgttgcagacataaatctcggcgactcggccccgtctcctgagggtcctgctggtgaccggcctggaccttggccctacagccctggaggccgctgctgaccagcactgaccccgacctcagagagtactcgcaggggcgctggctgcactcaagaccctcgagattaacggtgctaaccccgtctgctcctccctcccgcagagactggggcctggactggacatgagagccccttggtgccacagagggctgtgtcttactagaaacaacgcaaacctctccttcctcagaatagtgatgtgttcgacgttttatcaaaggccccctttctatgttcatgttagttttgctccttctgtgtttttttctgaaccatatccatgttgctgacttttccaaataaaggttttcactcctctaaaaaaaaaaaaaaaaaaaaa

SEQ ID NO: 5 - DNA, 2306 bp, Homo sapiens
gagcttgagaattgctcctgccctgggaagaggctcagcacagaaagaggaaggacagcacagctgacagccgtgctcagagagtttctggatcctaggcttatctccacagaggagaacacacaagcagcagagaccatgggaaccctctcagcccctccctgcacacagcgcatcaaatggaaggggctcctgctcacagcatcacttttaaacttctggaacctgcccaccactgcccaagtcacgattgaagccgagccaaccaaagtttccgaggggaaggatgttcttctacttgtccacaatttgccccagaatcttaccggctacatctggtacaaagggcaaatgagggacctctaccattacattacatcatatgtagtagacggtgaaataattatatatgggcctgcatatagtggacgagaaacagcatattccaatgcatccctgctgatccagaatgtcacccgggaggacgcaggatcctacaccttacacatcataaagggagatgatgggactagaggagtaactggacgtttcaccttcaccttacacctggagactcctaagccctccatctccagcagcaacttaaatcccagggagaccatggaggctgtgagcttaacctgtgaccctgagactccagacgcaagctacctgtggtggatgaatggtcagagcctccctatgactcacagcttgaagctgtccgaaaccaacaggaccctctttctattgggtgtcacaaagtatactgcaggaccctatgaatgtgaaatacggaacccagtgagtgccagccgcagtgacccagtcaccctgaatctcctcccgaagctgcccaagccctacatcaccatcaacaacttaaaccccagggagaataaggatgtcttaaacttcacctgtgaacctaagagtgagaactacacctacatttggtggctaaatggtcagagcctcccggtcagtcccagggtaaagcgacccattgaaaacaggatcctcattctacccagtgtcacgagaaatgaaacaggaccctatcaatgtgaaatacgggaccgatatggtggcatccgcagtgacccagtcaccctgaatgtcctctatggtccagacctccccagaatttacccttcattcacctattaccgttcaggagaagtcctctacttgtcctgttctgcggactctaacccaccggcacagtattcttggacaattaatgaaaagtttcagctaccaggacaaaagctctttatccgccatattactacaaagcatagcgggctctatgtttgctctgttcgtaactcagccactggcaaggaaagctccaaatccatgacagtcgaagtctctggtaagtggatcccagcatcgttggcaatagggttttaggtggagtctatctggcattcagagaagagtcaggaaaacaattgtattcccagcctgtgtcccatgggcacaagcaaatcccaaattctcctcctgaaccctccaaatttgtctaagaacttcgaaaactttaacaaacaggctgatatcttcataatattcccagcctagaccaagcaggaagaacattgatttcattgaaataattgataataatgaagataatgtttttatgatttttatttgaaaatttgctgattctttaaatggtttgttttctacattgatggaatttttctcttttaatctatctacagcttatagcagttcaataaactatacttctgggaaccgtaattgaaacatttacttttgctttctacctgactgccccagaattgggcaactattcatgagaattgatatgtttatggtaatacacatatttgcacaagtacagtaacaatctgctttctttgtaacatgacacatttgaaatcattggttatattaccaatgctttgattcggatgttatattaaaaacatagatagaatgaaccaatatgaactgcaggcaaagtctgaagtcagccttggtttggcttcctattctcaagaggtttgtgaagatttaatctcagattccttataaaaacttagagaaaagaaaattttagaagacagcctacatggtccattgctactcttgctgcacttatgtaaacaatcagaccacatttgaagaaactccacctattttgcaaacaaacttattctactgaaattatcattggtaaaagtagagatgcccatagagggaaaaattatgtggaaaataaaaactgtagtatacctaaaaaaaaaaaaaaaaaaaaaaaaaaaaaa

SEQ ID NO: 6 - DNA, 2883 bp, Homo sapiens
acgctgctccttcggtttccctgaaacctaacccgccctggggaggcgcgcagcagaggctccgattcggggcaggtgagaggctgactttctctcggtgcgtccagtggagctctgagtttcgaatcggcggcggcggattccccgcgcgcccggcgtcggggcttccaggaggatgcggagccccagcgcggcgtggctgctgggggccgccatcctgctagcagcctctctctcctgcagtggcaccatccaaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattgagttttccaggtcctcagatgggaattgcacagtaggatgtggaacctgtttaatgttatgaggacgtgtctgttatttcctaatcaaaaaggtctcaccacataccatgtggatgcagcacctctcaggattgctaggagctcccctgtttgcatgagaaaagtagtcccccaaattaacatcagtgtctgtttcagaatctctctactcagatgaccccagaaactgaaccaacagaagcagacttttcagaagatggtgaagacagaaacccagtaacttgcaaaaagtagacttggtgtgaagactcacttctcagctgaaattatatatatacacatatatatattttacatctgggatcatgatagacttgttagggcttcaaggccctcagagatgatcagtccaactgaacgaccttacaaatgaggaaaccaagataaatgagctgccagaatcaggtttccaatcaacagcagtgagttgggattggacagtagaatttcaatgtccagtgagtgaggttcttgtaccacttcatcaaaatcatggatcttggctgggtgcggtgcctcatgcctgtaatcctagcactttgggaggctgaggcaggcaatcacttgaggtcaggagttcgagaccagcctggccatcatggcgaaacctcatctctactaaaaatacaaaagttaaccaggtgtgtggtgcacgtttgtaatcccagttactcaggaggctgaggcacaagaattgagtatcactttaactcaggaggcagaggttgcagtgagccgagattgcaccactgcactccagcttgggtgataaaataaaataaaatagtcgtgaatcttgttcaaaatgcagattcctcagattcaataatgagagctcagactgggaacagggcccaggaatctgtgtggtacaaacctgcatggtgtttatgcacacagagatttgagaaccattgttctgaatgctgcttccatttgacaaagtgccgtgataatttttgaaaagagaagcaaacaatggtgtctcttttatgttcagcttataatgaaatctgtttgttgacttattaggactttgaattatttctttattaaccctctgagtttttgtatgtattattattaaagaaaaatgcaatcaggattttaaacatgtaaatacaaattttgtataacttttgatgacttcagtgaaattttcaggtagtctgagtaatagattgttttgccacttagaatagcatttgccacttagtattttaaaaaataattgttggagtatttattgtcagttttgttcacttgttatctaatacaaaattataaagccttcagagggtttggaccacatctctttggaaaatagtttgcaacatatttaagagatacttgatgccaaaatgactttatacaacgattgtatttgtgacttttaaaaataattattttattgtgtaattgatttataaataacaaaattttttttacaactta

SEQ ID NO: 7 - DNA, 3549 bp, Homo sapiens
acatctggcggctgccctcccttgtttccgctgcatccagacttcctcaggcggtggctggaggctgcgcatctggggctttaaacatacaaagggattgccaggacctgcggcggcggcggcggcggcgggggctggggcgcgggggccggaccatgagccgctgagccgggcaaaccccaggccaccgagccagcggaccctcggagcgcagccctgcgccgcggagcaggctccaaccaggcggcgaggcggccacacgcaccgagccagcgacccccgggcgacgcgcggggccagggagcgctacgatggaggcgctaatggcccggggcgcgctcacgggtcccctgagggcgctctgtctcctgggctgcctgctgagccacgccgccgccgcgccgtcgcccatcatcaagttccccggcgatgtcgcccccaaaacggacaaagagttggcagtgcaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcaacccagatgtggccaactacaacttcttccctcgcaagcccaagtgggacaagaaccagatcacatacaggatcattggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatgggaacgccgatggggagtactgcaagttccccttcttgttcaatggcaaggagtacaacagctgcactgataccggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgatgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacgctgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccctggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggcttccccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctgagctggccctggctcccacaggcccttcctctccactgccttcgatacaccgggcctggagaactagagaaggacccggaggggcctggcagccgtgccttcagctctacagctaatcagcattctcactcctacctggtaatttaagattccagagagtggctcctcccggtgcccaagaatagatgctgactgtactcctcccaggcgccccttccccctccaatcccaccaaccctcagagccacccctaaagagatactttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggccactccagacccctggcttttcactgctggctgccttagaacctttcttacattagcagtttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaatgtcaacaagtatgaataaagacacctactgagtggccgtgtttgccatctgttttagcagagcctagacaagggccacagacccagccagaagcggaaacttaaaaagtccgaatctctgctccctgcagggcacaggtgatggtgtctgctggaaaggtcagagcttccaaagtaaacagcaagagaacctcagggagagtaagctctagtccctctgtcctgtagaaagagccctgaagaatcagcaattttgttgctttattgtggcatctgttcgaggtttgcttcctctttaagtctgtttcttcattagcaatcatatcagttttaatgctactactaacaatgaacagtaacaataatatccccctcaattaatagagtgctttctatgtgcaaggcacttttcacgtgtcacctattttaacctttccaaccacataaataaaaaaggccattattagttgaaaaaaaaaaaaaaaaaaaaaaaaa

SEQ ID NO: 8 - DNA, 3045 bp, Homo sapiens
ggtttgcaggcggaggctacagctagggctgctcagtaactgccccaaccactaccacgtccccaagtgcaacgggacccacggaactacagggtcctgctgagctcaggagctgcacggaatggtggcagtcaccttgccagtgcagcaggcatggatcagactgcaagctaggggcacgaggcatcagttgggaagaggggacgcacagctaggcttgaggcctcttcatcgggatgtccccaggccccccagcccaggcccagcataaaggccgtgttggggggcccccctgacccaaggggggcttcatgcgccacgtgcaggcggagccgtctccatcctcagagccggaggctggcccttcacagcctccagtcaggcagggggccctccagggtggcctgctcatgggctacagcccagcagggggggcgacatcccccggggtctaccaggtatccatcttttcccctccggctggtacctctgagcctcatagggccctgaaacgacaagccccatccactgagggtccccgggagctgaagagaggccctgggctgggggccagagagggactaccccctgaagaaccatctactgtggggctcttaggcccagagggaccggggctgggactaggtgtggccagccagcatttctcccaccgtggcctctgtgttgtggaacagagaagtagtgtcacctcatcttggacttcaggggcctggagtcccccctgccccccatcaaatgcttcctgcaatactttgcacaccagagactgggcctccccagatccagggggacaggggtccctgggggagtccccagggccagcccctccaggccagctgcacacacttgacactgatttgcacagtcttgcacaaatagggggtaagagcccagtggctggggtgggcaatgggggtagcctctggcctagggagtcccctggcactgccaatgggcacagtcccgagcacacaccccctggccctggacccccaggcccctgccccaccaagcgaaggctgcttcctgctggagaagccccagatgtcagctctgaggaagaggggccagcccctcggaggcgccggggatccctgggccaccctactgctgccaacagttctgatgccaaagccacacccttctggagccacctgctgcctgggcccaaagagcctgttttggacccaacagactgcggtcccatggggcggaggctgaaaggagcccgtcgcctgaagctgagcccccttcgaagcctccggaaggggccaggcctgctgagcccccccagtgcctcccctgttcctacccctgctgtcagccgtaccctgctgggcaactttgaggaatcattgctgcgaggacgttttgcaccatctggccacattgagggcttcacagcagaaattggagctagtgggtcatactgcccccagcacgtcacgctgcctgtcactgtcacattctttgatgtttctgagcaaaatgccccggctcccttcctgggcatcgtggatctgaaccccttggggaggaagggttacagcgtgcccaaggtgggcaccgtccaagtgaccttatttaaccccaaccagactgtggtaaagatgttccttgtgacctttgacttctcggacatgcctgctgcccacatgaccttcctgcgccatcgcctctttttggtgcctgtgggtgaggagggaaatgctaaccccacccaccgcctcctctgctacttgctgcacctcaggttccggagctcccgctcaggccgcttaagcctgcatggagatatccgcctgcttttttcccgccggagcctggagctggacacagggctcccctacgaactgcaggctgtgaccgaggcccctcataatccacgttattcacctttgccctgattgccagcactctgaacccatgcgggctaatgacctgcccatcctgctccatcttagagaacatatatggagagacagcaagagacccttcaggcttgaattaaagccctcaccatgctcacgcccaaatggattatttgggtgtttaaagcttctgattcttactacaccctgccctacttcgggtactccatgtgcctgtcccctcccttgggtttcccaggccagcttaggtagtagggaggaactggagctaccctgagattttctcaagttcccaggcaagttatgccagcgttgcctccctgtcctgggcaggggccacttgttattttatttatttttaatttataatttattcaaattggattgccttggtaacctcccacacctgataattggcatcactcctccccgcttcccactctcagatattgctccaacctcaggagttgaagaggcttatgcggtgggggcaggaggagaactgctttccctcagctgagggaagaggggctattccagagggactgagtcagtagccaaagactcagcttcccctgtccttccccagtcccttcacttcccctaccctctgacctatctctgaaagccaagttatgcgtatgtgtgtgtgcacaagcttgtctttgtgtggtatgtgtgtgtgagtgtgcatgtatgcacacacacaggggttaaccacccctcacctagggctccagactccagttgtccctctcctcctacctgtgtctccttgttttggggtcctgactgaagaaggtgtccagggggcagagtcagggccaagcactggggtgcctcctctcacctggccagactctgacccacctacctcagctggggtgaggggcacccctcaaactcagtcatgtggttccaaactaccccattccccactccagactctgacccagcctcagtcctaactcctggggctgggctgaggggaacaagcatttgctgaaacttgaaaaaacaaagcaaatcaaaaacaggaaaaaattgtacctggtacttttttttagaaaaaaagattaaaaaagaaagaataaattcttgtttggaaacttgaaaaaaaaaaaaaaaaaaa

SEQ ID NO: 9 - DNA, 2964 bp, Homo sapiens
cagagccacgggcgcccgccccgccccgcgccgccccgcgccggctccgcagctcgcgcccgcccgcctgccggcccgcccggcgccgggccatggcgctgctgcgggatgtgtcgctgcaggacccgcgggaccgcttcgagctgctgcagcgcgtgggggccgggacctatggcgacgtctacaaggcccgcgacacggtcacgtccgaactggccgccgtgaagatagtcaagctagacccaggggacgacatcagctccctccagcaggaaatcaccatcctgcgtgagtgccgccaccccaatgtggtggcctacattggcagctacctcaggaatgaccgcttgtggatctgcatggagttctgcggagggggctccctgcaggagatttaccatgccactgggcccctggaggagcggcagattgcctacgtctgccgagaggcactgaaggggctccaccacctgcattctcaggggaagatccacagagacatcaagggagccaaccttctcctcactctccagggagatgtcaaactggctgactttggggtgtcaggcgagctgacagcgtctgtggccaagaggaggtctttcattgggactccctactggatggctcccgaggtggctgctgtggagcgcaaaggtggctacaatgagctatgtgacgtctgggccctgggcatcactgccattgagctgggcgagctgcagccccctctgttccacctgcaccccatgagggccctgatgctcatgtcgaagagcagcttccagccgcccaaactgagagataagactcgctggacccagaatttccaccactttctcaaactggccctgaccaagaatcctaagaagaggccgacagcagagaagctcctgcagcacccgttcacgactcagcagctccctcgggccctcctcacacagctgctggacaaagccagtgaccctcatctggggaccccctcccctgaggactgtgagctggagacctatgacatgtttccagacaccattcactcccgggggcagcacggcccagccgagaggaccccctcggagatccagtttcaccaggtgaaatttggcgccccacgcaggaaggaaactgacccactgaatgagccgtgggaggaagagtggacactactgggaaaggaagagttgagtgggagcctgctgcagtcggtccaggaggccctggaggaaaggagtctgactattcggtcagcctcagaattccaggagctggactccccagacgataccatgggaaccatcaagcgggccccgttcctagggccactccccactgaccctccagcagaggagcctctgtccagtcccccaggaaccctgcccccacctccttcaggccccaacagctccccactgctgcccacggcctgggccaccatgaagcagcgggaggatcctgagaggtcatcctgccacgggctccccccaactcccaaggtgcatatgggcgcctgcttctccaaggtcttcaatggctgccccctgcggatccacgctgctgtcacctggattcaccctgttactcgggaccagttcctggtggtaggggccgaggaaggcatctacacactcaacctgcatgaactgcatgaggatacgctggagaagctgatttcacatcgctgctcctggctctactgcgtgaacaacgtgctgctgtcactctcagggaaatccacgcacatctgggcccatgacctcccaggcctgtttgagcagcggaggctacagcaacaggttcccctctccatccccaccaaccgcctcacccagcgcatcatccccaggcgctttgctctgtccaccaagattcctgacaccaaaggctgcttgcagtgtcgtgtggtgcggaacccctacacgggtgccaccttcctgctggccgccctgcccaccagcctgctcctgctgcagtggtatgagccgctgcagaagtttctgctgctgaagaacttctccagccctctgcccagcccagctgggatgctggagccgctggtgctggatgggaaggagctgccgcaggtgtgtgttggggccgaggggcctgaggggcccggctgccgcgtcctgttccatgtcctgcccctggaggctggcctgacgcccgacatcctcatcccacctgaggggatcccaggctcggcccagcaggtgatccaggtggacagggacacaatcctagtcagctttgaacgctgtgtgaggattgtcaacatgcagggcgagcccacggccacactggcacctgagctgacctttgatttccccatcgagactgtggtgtgcctgcaggacagtgtgctggccttctggagccatgggatgcaaggccgaagcctggataccaatgaggtgacccaggagatcacagatgaaacaaggatcttccgagtgcttggggcccacagagacatcatcctggagagcattcccactgacaacccagaggcgcacagcaacctctacatcctcacgggccaccagagcacctactaagagcagcgggcctgtccaggggctccccgccccaccccacgccttagctgcaggcccttttgggcaaaggggcccatcctagaccagaggagcccaggccctggccctgctggggctgaaggtcagaagtaatcctgagaaatgtttcaggcctggggagggaggggagcccccgacgcctctgcaataactggaccagggggagctgctgtcactcccccatccccgaggcagcccagtccctagtgcccaaggcagggaccctgggcctgggccatccattccattttgttccacatttcctttctactctttctgccaagagcctgcccctgcatttgtcctgggaaacacggtatttaagagagaactatattggtattaaagctggtttgttttaaaaaaaaaa

SEQ ID NO: 10 - DNA, 5925 bp, Homo sapiens
gcgctgcccgcctcgtccccacccccccaacccccgcgcccgccctcggacagtccctgctcgcccgcgcgctgcagccccatctcctagcggcagcccaggcgcggagggagcgagtccgccccgaggtaggtccaggacgggcgcacagcagcagccgaggctggccgggagagggaggaagaggatggcagggccacgccccagcccatgggccaggctgctcctggcagccttgatcagcgtcagcctctctgggaccttggcaaaccgctgcaagaaggccccagtgaagagctgcacggagtgtgtccgtgtggataaggactgcgcctactgcacagacgagatgttcagggaccggcgctgcaacacccaggcggagctgctggccgcgggctgccagcgggagagcatcgtggtcatggagagcagcttccaaatcacagaggagacccagattgacaccaccctgcggcgcagccagatgtccccccaaggcctgcgggtccgtctgcggcccggtgaggagcggcattttgagctggaggtgtttgagccactggagagccccgtggacctgtacatcctcatggacttctccaactccatgtccgatgatctggacaacctcaagaagatggggcagaacctggctcgggtcctgagccagctcaccagcgactacactattggatttggcaagtttgtggacaaagtcagcgtcccgcagacggacatgaggcctgagaagctgaaggagccctggcccaacagtgacccccccttctccttcaagaacgtcatcagcctgacagaagatgtggatgagttccggaataaactgcagggagagcggatctcaggcaacctggatgctcctgagggcggcttcgatgccatcctgcagacagctgtgtgcacgagggacattggctggcgcccggacagcacccacctgctggtcttctccaccgagtcagccttccactatgaggctgatggcgccaacgtgctggctggcatcatgagccgcaacgatgaacggtgccacctggacaccacgggcacctacacccagtacaggacacaggactacccgtcggtgcccaccctggtgcgcctgctcgccaagcacaacatcatccccatctttgctgtcaccaactactcctatagctactacgagaagcttcacacctatttccctgtctcctcactgggggtgctgcaggaggactcgtccaacatcgtggagctgctggaggaggccttcaatcggatccgctccaacctggacatccgggccctagacagcccccgaggccttcggacagaggtcacctccaagatgttccagaagacgaggactgggtcctttcacatccggcggggggaagtgggtatataccaggtgcagctgcgggcccttgagcacgtggatgggacgcacgtgtgccagctgccggaggaccagaagggcaacatccatctgaaaccttccttctccgacggcctcaagatggacgcgggcatcatctgtgatgtgtgcacctgcgagctgcaaaaagaggtgcggtcagctcgctgcagcttcaacggagacttcgtgtgcggacagtgtgtgtgcagcgagggctggagtggccagacctgcaactgctccaccggctctctgagtgacattcagccctgcctgcgggagggcgaggacaagccgtgctccggccgtggggagtgccagtgcgggcactgtgtgtgctacggcgaaggccgctacgagggtcagttctgcgagtatgacaacttccagtgtccccgcacttccgggttcctctgcaatgaccgaggacgctgctccatgggccagtgtgtgtgtgagcctggttggacaggcccaagctgtgactgtcccctcagcaatgccacctgcatcgacagcaatgggggcatctgtaatggacgtggccactgtgagtgtggccgctgccactgccaccagcagtcgctctacacggacaccatctgcgagatcaactactcggcgatccacccgggcctctgcgaggacctacgctcctgcgtgcagtgccaggcgtggggcaccggcgagaagaaggggcgcacgtgtgaggaatgcaacttcaaggtcaagatggtggacgagcttaagagagccgaggaggtggtggtgcgctgctccttccgggacgaggatgacgactgcacctacagctacaccatggaaggtgacggcgcccctgggcccaacagcactgtcctggtgcacaagaagaaggactgccctccgggctccttctggtggctcatccccctgctcctcctcctcctgccgctcctggccctgctactgctgctatgctggaagtactgtgcctgctgcaaggcctgcctggcacttctcccgtgctgcaaccgaggtcacatggtgggctttaaggaagaccactacatgctgcgggagaacctgatggcctctgaccacttggacacgcccatgctgcgcagcgggaacctcaagggccgtgacgtggtccgctggaaggtcaccaacaacatgcagcggcctggctttgccactcatgccgccagcatcaaccccacagagctggtgccctacgggctgtccttgcgcctggcccgcctttgcaccgagaacctgctgaagcctgacactcgggagtgcgcccagctgcgccaggaggtggaggagaacctgaacgaggtctacaggcagatctccggtgtacacaagctccagcagaccaagttccggcagcagcccaatgccgggaaaaagcaagaccacaccattgtggacacagtgctgatggcgccccgctcggccaagccggccctgctgaagcttacagagaagcaggtggaacagagggccttccacgacctcaaggtggcccccggctactacaccctcactgcagaccaggacgcccggggcatggtggagttccaggagggcgtggagctggtggacgtacgggtgcccctctttatccggcctgaggatgacgacgagaagcagctgctggtggaggccatcgacgtgcccgcaggcactgccaccctcggccgccgcctggtaaacatcaccatcatcaaggagcaagccagagacgtggtgtcctttgagcagcctgagttctcggtcagccgcggggaccaggtggcccgcatccctgtcatccggcgtgtcctggacggcgggaagtcccaggtctcctaccgcacacaggatggcaccgcgcagggcaaccgggactacatccccgtggagggtgagctgctgttccagcctggggaggcctggaaagagctgcaggtgaagctcctggagctgcaagaagttgactccctcctgcggggccgccaggtccgccgtttccacgtccagctcagcaaccctaagtttggggcccacctgggccagccccactccaccaccatcatcatcagggacccagatgaactggaccggagcttcacgagtcagatgttgtcatcacagccaccccctcacggcgacctgggcgccccgcagaaccccaatgctaaggccgctgggtccaggaagatccatttcaactggctgcccccttctggcaagccaatggggtacagggtaaagtactggattcagggtgactccgaatccgaagcccacctgctcgacagcaaggtgccctcagtggagctcaccaacctgtacccgtattgcgactatgagatgaaggtgtgcgcctacggggctcagggcgagggaccctacagctccctggtgtcctgccgcacccaccaggaagtgcccagcgagccagggcgtctggccttcaatgtcgtctcctccacggtgacccagctgagctgggctgagccggctgagaccaacggtgagatcacagcctacgaggtctgctatggcctggtcaacgatgacaaccgacctattgggcccatgaagaaagtgctggttgacaaccctaagaaccggatgctgcttattgagaaccttcgggagtcccagccctaccgctacacggtgaaggcgcgcaacggggccggctgggggcctgagcgggaggccatcatcaacctggccacccagcccaagaggcccatgtccatccccatcatccctgacatccctatcgtggacgcccagagcggggaggactacgacagcttccttatgtacagcgatgacgttctacgctctccatcgggcagccagaggcccagcgtctccgatgacactggctgcggctggaagttcgagcccctgctgggggaggagctggacctgcggcgcgtcacgtggcggctgcccccggagctcatcccgcgcctgtcggccagcagcgggcgctcctccgacgccgaggcgccccacgggcccccggacgacggcggcgcgggcgggaagggcggcagcctgccccgcagtgcgacacccgggccccccggagagcacctggtgaatggccggatggactttgccttcccgggcagcaccaactccctgcacaggatgaccacgaccagtgctgctgcctatggcacccacctgagcccacacgtgccccaccgcgtgctaagcacatcctccaccctcacacgggactacaactcactgacccgctcagaacactcacactcgaccacactgcccagggactactccaccctcacctccgtctcctcccacgactctcgcctgactgctggtgtgcccgacacgcccacccgcctggtgttctctgccctggggcccacatctctcagagtgagctggcaggagccgcggtgcgagcggccgctgcagggctacagtgtggagtaccagctgctgaacggcggtgagctgcatcggctcaacatccccaaccctgcccagacctcggtggtggtggaagacctcctgcccaaccactcctacgtgttccgcgtgcgggcccagagccaggaaggctggggccgagagcgtgagggtgtcatcaccattgaatcccaggtgcacccgcagagcccactgtgtcccctgccaggctccgccttcactttgagcactcccagtgccccaggcccgctggtgttcactgccctgagcccagactcgctgcagctgagctgggagcggccacggaggcccaatggggatatcgtcggctacctggtgacctgtgagatggcccaaggaggagggccagccaccgcattccgggtggatggagacagccccgagagccggctgaccgtgccgggcctcagcgagaacgtgccctacaagttcaaggtgcaggccaggaccactgagggcttcgggccagagcgcgagggcatcatcaccatagagtcccaggatggaggacccttcccgcagctgggcagccgtgccgggctcttccagcacccgctgcaaagcgagtacagcagcatcaccaccacccacaccagcgccaccgagcccttcctagtggatgggctgaccctgggggcccagcacctggaggcaggcggctccctcacccggcatgtgacccaggagtttgtgagccggacactgaccaccagcggaacccttagcacccacatggaccaacagttcttccaaacttgaccgcaccctgccccacccccgccacgtcccactaggcgtcctcccgactcctctcccggagcctcctcagctactccatccttgcacccctgggggcccagcccacccgcatgcacagagcaggggctaggtgtctcctgggaggcatgaagggggcaaggtccgtcctctgtgggcccaaacctatttgtaaccaaagagctgggagcagcacaaggacccagcctttgttctgcacttaataaatggttttgctactgctaaaaaaa

SEQ ID NO: 11 - DNA, 3125 bp, Homo sapiens
agggacttacccaaggtcacgcagcgagcccggtccccctgcgttccccggggagcgctgagccgggacgcggcggtggggtggggaaggggagtggcgcggccctgcggggtgaggctgccgtttgctgagtgtccggcaggggtctgctcgctgccagcccggcccctcctcagagcagctgccgcagcccgaggatgtcggaggagatcatcacgccggtgtactgcactggggtgtcagcccaagtgcagaagcagcgggccagggagctgggcctgggccgccatgagaatgccatcaagtacctgggccaggattatgagcagctgcgggtgcgatgcctgcagagtgggaccctcttccgtgatgaggccttccccccggtaccccagagcctgggttacaaggacctgggtcccaattcctccaagacctatggcatcaagtggaagcgtcccacggaactgctgtcaaacccccagttcattgtggatggagctacccgcacagacatctgccagggagcactgggggactgctggctcttggcggccatcgcctccctcactctcaacgacaccctcctgcaccgagtggttccgcacggccagagcttccagaatggctatgccggcatcttccatttccagctgtggcaatttggggagtgggtggacgtggtcgtggatgacctgctgcccatcaaggacgggaagctagtgttcgtgcactctgccgaaggcaacgagttctggagcgccctgcttgagaaggcctatgccaaggtaaatggcagctacgaggccctgtcagggggcagcacctcagagggctttgaggacttcacaggcggggttaccgagtggtacgagttgcgcaaggctcccagtgacctctaccagatcatcctcaaggcgctggagcggggctccctgctgggctgctccatagacatctccagcgttctagacatggaggccatcactttcaagaagttggtgaagggccatgcctactctgtgaccggggccaagcaggtgaactaccgaggccaggtggtgagcctgatccggatgcggaacccctggggcgaggtggagtggacgggagcctggagcgacagctcctcagagtggaacaacgtggacccatatgaacgggaccagctccgggtcaagatggaggacggggagttctggatgtcattccgagacttcatgcgggagttcacccgcctggagatctgcaacctcacacccgacgccctcaagagccggaccatccgcaaatggaacaccacactctacgaaggcacctggcggcgggggagcaccgcggggggctgccgaaactacccagccaccttctgggtgaaccctcagttcaagatccggctggatgagacggatgacccggacgactacggggaccgcgagtcaggctgcagcttcgtgctcgcccttatgcagaagcaccgtcgccgcgagcgccgcttcggccgcgacatggagactattggcttcgcggtctacgaggtccctccggagctggtgggccagccggccgtacacttgaagcgtgacttcttcctggccaatgcgtctcgggcgcgctcagagcagttcatcaacctgcgagaggtcagcacccgcttccgcctgccacccggggagtatgtggtggtgccctccaccttcgagcccaacaaggagggcgacttcgtgctgcgcttcttctcagagaagagtgctgggactgtggagctggatgaccagatccaggccaatctccccgatgagcaagtgctctcagaagaggagattgacgagaacttcaaggccctcttcaggcagctggcaggggaggacatggagatcagcgtgaaggagttgcggacaatcctcaataggatcatcagcaaacacaaagacctgcggaccaagggcttcagcctagagtcgtgccgcagcatggtgaacctcatggatcgtgatggcaatgggaagctgggcctggtggagttcaacatcctgtggaaccgcatccggaattacctgtccatcttccggaagtttgacctggacaagtcgggcagcatgagtgcctacgagatgcggatggccattgagtcggcaggcttcaagctcaacaagaagctgtacgagctcatcatcacccgctactcggagcccgacctggcggtcgactttgacaatttcgtttgctgcctggtgcggctagagaccatgttccgatttttcaaaactctggacacagatctggatggagttgtgacctttgacttgtttaagtggttgcagctgaccatgtttgcatgaggcagggactcggtcccccttgccgtgctcccctccctcctcgtctgccaagcctcgcctcctaccacaccacaccaggccaccccagctgcaagtgccttccttggagcagagaggcagcctcgtcctcctgtcccctctcctcccagccaccatcgttcatctgctccgggcagaactgtgtggcccctgcctgtgccagccatgggctcgggatggactccctgggccccacccattgccaagccaggaaggcagctttcgcttgttcctgcctcgggacagccccgggtttccccagcatcctgatgtgtcccctctccccacttcagaggccacccactcagcaccaccggcctggccttgcctgcagactataaactataaccactagctcgacacagtctgcagtccaggcgtgtggagccgcctcccggctcggggaggccccggggctgggaacgcctgtgccttcctgcgccgaagccaacgccccctctgtccttccctggccctgctgccgaccaggagctgcccagcctgtgggcggtcggccttccctccttcgctccttttttatattagtgattttaaaggggactcttcagggacttgtgtactggttatgggggtgccagaggcactaggcttggggtggggaggtcccgtgttccatatagaggaaccccaaataataaaaggccccacatctgtctgtgaaaaaaaaaaaaaaaaaaaaaaaaaaaaaaaaaaaaaaaaaaaaaaaaaaaaaaaaaaaaaaaa

SEQ ID NO: 12 - DNA, 1172 bp, Homo sapiens
gcgatgcgggcgcccccggcgggcggccccggcgggcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaagaggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcctcccctcactaagaagacccaaacctctgcataatgggatttgggctttggtacaagaactgtgacccccaaccctgataaaagagatggaaggaaaaaaaaaaaaaaaaaaaaaaaaaaaaaaaaaaaaaaaaaaaaaaaaaaaaaaa

(19) United States
(12) Patent Application Publication
(10) Pub. No.: US 2014/0080737 A1
(43) Pub. Date: Mar. 20, 2014

(54) GENE EXPRESSION PROFILE FOR THERAPEUTIC RESPONSE TO VEGF INHIBITORS

(71) Applicant: Patrick J. Muraca, Pittsfield, MA (US)

(72) Inventor: Patrick J. Muraca, Pittsfield, MA (US)

(73) Assignee: NUCLEA BIOTECHNOLOGIES, INC., Pittsfield, MA (US)

(21) Appl. No.: 14/110,456

(22) PCT Filed: Apr. 13, 2012
(86) PCT No.: PCT/US12/33416

§ 371 Date: Nov. 15, 2013

Related U.S. Application Data
(60) Provisional application No. 61/475,850, filed on Apr. 15, 2011.

(51) Int. Cl. G01N 33/68

(52) U.S. Cl. CPC G01N 33/6893
USPC 506/9; 435/7.9; 530/389.1; 506/16; 435/7.4

(57) ABSTRACT

The invention provides gene expression profiles (GEPs), protein expression profiles (PEPs) as well as gene/protein expression profiles (GPEPs) and methods for using them to identify metastatic breast cancer patients who are likely to respond to therapy with a VEGF inhibitor. The present invention allows a treatment provider to identify those patients who are most likely to respond to such treatment, and to initiate and/or adjust treatment options for such patients accordingly.

### CROSS REFERENCE TO RELATED APPLICATIONS

This application claims the benefit of U.S. Provisional Patent Application No. 61/475,850, filed Apr. 15, 2011, entitled “Gene Expression Profile For Therapeutic Response to VEGF Inhibitors” the contents, each of which is incorporated by reference in its entirety.

### REFERENCE TO SEQUENCE LISTING

The present application is being filed along with a Sequence Listing in electronic format. The Sequence Listing is provided as a file entitled NUC058SEQLST.txt created on Apr. 12, 2012 which is 46,700 bytes in size. The information in electronic format of the sequence listing is incorporated herein by reference in its entirety.

### FIELD OF THE INVENTION

The invention relates to compositions and methods for determining the therapeutic efficacy of VEGF inhibitors in treating metastatic breast cancer patients.

### BACKGROUND OF THE INVENTION

Vascular endothelial growth factor (VEGF)-mediated angiogenesis is thought to play a critical role in tumor growth and metastasis. Consequently, anti-VEGF therapies are being actively investigated as potential anti-cancer treatments, either as alternatives or adjuncts to conventional chemo or radiation therapy. Among the techniques used to block the VEGF pathway are: 1) neutralizing monoclonal antibodies against VEGF or its receptor, 2) small molecule tyrosine kinase inhibitors of VEGF receptors, and 3) soluble VEGF receptors which act as decoy receptors for VEGF. An anti-VEGF monoclonal antibody, bevacizumab (Avastin®), has been approved by the FDA as first line therapy in metastatic colorectal carcinoma in combination with other chemotherapeutic agents. However, many challenges still remain, and the role of anti-VEGF therapy in the treatment of other solid tumors remains to be elucidated.

Angiogenesis has been an appealing target for anticancer drugs for 30 years, but it is only recently that this promise has been realized. There are now over 30 angiogenesis inhibitors currently in clinical trials for the treatment of malignancy. These drugs appear to have a cytostatic rather than cytotoxic effect, leading to tumor dormancy. The available data suggest that anti-angiogenic drugs work best in conjunction with chemotherapy. Their development also involves the identification and management of a new range of patient responsiveness.

The present invention provides methods and compositions, including gene and protein expression profiles, for the evaluation of responsiveness of cancer patients to VEGF inhibitors.

### SUMMARY OF THE INVENTION

The present invention is based on a study of patients that have developed metastatic breast cancer. The invention provides gene expression profiles (GEPs), protein expression profiles (PEPs) as well as gene/protein expression profiles (GPEPs) and methods for using them to identify those patients who are likely to respond to treatment with a VEGF inhibitor. The present invention allows a treatment provider to stratify patients; that is, to identify those patients most likely to respond to and benefit from therapy with a VEGF inhibitor, and those that are less likely to respond to treatment with a VEGF inhibitor, but may benefit from alternative therapies.

In one aspect, the present invention provides gene expression profiles (GEPs), also referred to as “gene signatures,” that are indicative of the likelihood that a patient's metastatic breast cancer will respond to treatment with a VEGF inhibitor. In one embodiment, the gene expression profile (GEP) comprises at least one, and preferably a plurality, of genes selected from the group consisting of genes encoding the following proteins: VEGF, S100A3, PIGO, COL6A1, PSG1, F2RL1, MMP2, KIAA1539, MAP4K2, ITGB4 and CAPN1. In an alternate embodiment, the present invention provides a GEP comprising at least one, and preferably a plurality, of the genes encoding the following proteins: VEGF, MMP2, KIAA1539, MAP4K2, ITGB4 and CAPN1. In yet another embodiment, the gene expression profile (GEP) comprises at least one, and preferably a plurality, of genes selected from the group consisting of genes encoding the following proteins: VEGF, S100A3, PIGO, COL6A1, PSG1 and F2RL1. All of these genes are up-regulated (overexpressed) in the tumor tissue and sera of patients whose metastatic breast cancer is likely to respond to VEGF-inhibitor therapy.

In one aspect, the present invention provides protein expression profiles (PEPs) that are indicative of the likelihood that a patient's metastatic breast cancer is likely to respond to therapy with a VEGF inhibitor. The protein expression profiles comprise proteins that are differentially expressed in breast a cancer patient whose disease has metastasized, and is likely to respond to therapy with a VEGF inhibitor. The present protein expression profile (PEP) comprises at least one, and preferably a plurality, of proteins selected from the group consisting of: VEGF, S100A3, PIGO, COL6A1, PSG1, F2RL1, MMP2, KIAA1539, MAP4K2, ITGB4 and CAPN1. In an alternate embodiment, the present invention further provides a further PEP comprising at least one of the proteins from the group consisting of VEGF, S100A3, PIGO, COL6A1, PSG1 and F2RL1. In yet another embodiment, the present invention provides a PEP comprising at least one of the proteins from the group consisting of VEGF, MMP2, KIAA1539, MAP4K2, ITGB4 and CAPN1. All of these proteins are up-regulated in the tumor tissue and sera of patients whose metastatic breast cancer is likely to respond to VEGF-inhibitor therapy.

The present gene and protein expression profiles further may include reference or control genes and the proteins expressed thereby. The currently preferred reference genes are beta-actin (ACTB), glyceraldehyde-3-phosphate dehydrogenase (GAPDH), beta glucoronidase (GUSB), large ribosomal protein (RPLP0) and/or transferrin receptor (TRFC).

In one embodiment a method is provided of determining if a patient's metastatic breast cancer is likely to respond to VEGF-inhibitor therapy. The method comprises obtaining a tumor and/or serum sample from the patient, determining the gene and/or protein expression profile of the sample, and determining from the gene or protein expression profile whether at least about 2, and preferably a plurality, of the genes or encoded proteins selected from the group consisting of: VEGF, S100A3, PIGO, COL6A1, PSG1, F2RL1, MMP2, KIAA1539, MAP4K2, ITGB4 and CAPN1 are differentially expressed, specifically upregulated, in the sample. In alternate embodiments, the method comprises obtaining a tumor and/or serum sample from the patient, determining the gene and/or protein expression profile of the sample, and determining from the gene or protein expression profile whether at least about 2, preferably 4 and most preferably all six of the genes or encoded proteins selected from the group consisting of: VEGF, S100A3, PIGO, COL6A1, PSG1 and F2RL1; or the group consisting of: VEGF, MMP2, KIAA1539, MAP4K2, ITGB4 and CAPN1; are differentially expressed, specifically upregulated, in the sample. From this information, the treatment provider can ascertain whether the patient's disease is likely to respond to treatment with a VEGF inhibitor, and tailor the patient's treatment accordingly.

The present invention further comprises assays for determining the gene and/or protein expression profile in a patient's sample, and instructions for using the assay. The assay may be based on detection of nucleic acids (e.g., using nucleic acid probes specific for the nucleic acids of interest) or proteins or peptides (e.g., using antibodies specific for the proteins/peptides of interest). In a preferred embodiment, the assay comprises an immunohistochemistry (IHC) test in which tissue or serum samples are contacted with antibodies specific for the proteins/peptides identified in the GPEP as being indicative of the likelihood that the patient will respond to treatment with a VEGF inhibitor.

Practice of the present invention allows the patient and caregiver to make better clinical decisions, e.g., frequency of monitoring, administration of adjuvant radiation or chemotherapy, or design of an appropriate therapeutic regimen.

The details of various embodiments of the invention are set forth in the description below. Other features, objects, and advantages of the invention will be apparent from the description and from the claims.

### DETAILED DESCRIPTION OF THE INVENTION

Described herein are compositions and methods for employing gene and protein expression profiles in prognosis or prediction of the likelihood a subject afflicted with metastatic breast cancer will respond to treatment with a VEGF inhibitor.

The term “metastatic” describes a cancer that has spread to distant organs from the original tumor site. Metastatic breast cancer is the most advanced stage (stage IV) of breast cancer. Cancer cells have spread past the breast and axillary (underarm) lymph nodes to other areas of the body where they continue to grow and multiply. Breast cancer has the potential to spread to almost any region of the body. The most common regions that breast cancer spreads to are: the same breast as the primary tumor or the other breast, chest wall, lymph nodes, bone, lung, liver and brain.

Breast cancer often begins in the breast ducts as ductal carcinoma in situ (DCIS). Once out of the breast, cancer often spreads first to the axillary (underarm) lymph nodes. One or more of the lymph nodes are usually removed during breast surgery to determine whether the nodes are involved. In some cases, breast cancer may spread to other regions of the body without involving the axillary lymph nodes. If the cancerous tumor is located in the medial portion of the breast, it may spread to the internal mammary nodes which are located between the ribs and beneath the sternum. In some cases, cancer may spread through the bloodstream without being detected in the lymphatic system. Metastatic breast cancer may also occur from a recurrence of breast cancer after initial treatment.

Positive treatment outcomes for metastatic breast cancer depend highly on early detection and prompt therapeutic intervention. Most early detections are achieved with the use of physical examinations or imaging technologies such as mammography, MRI and the like. However, these techniques do not provide any guidance as to which therapeutic regimen is likely to be effective. Consequently, patients experiencing metastatic breast cancer do not always receive the most beneficial therapy as early as possible, resulting in poorer long-term outcome measures such as remission or survival. The GEPs and PEPs (collectively the GPEPs) of the present invention provide the clinician with a prognostic tool capable of providing valuable information that can positively affect management of the disease. According to the present invention, oncologists can assay the suspect tissue/serum for the presence of members of the novel GPEP, and can identify with a high degree of accuracy those patients whose condition is likely to respond to therapy with a VEGF inhibitor. This information, taken together with other available clinical information including imaging data, allows more effective management of the disease.

In a preferred aspect of the invention, the expression of genes or proteins in a tumor tissue and/or serum sample from a patient is assayed using tissue array, immunohistochemistry, ELISA or other assay technique to identify the expression of genes or proteins in the present GPEP. Metastatic breast cancer tumors may occur, for example, in breast tissue (either the same breast as the original occurrence or the other breast), or in lymph node, chest wall, bone, lung, liver, or brain tissue or example. The gene or protein expression profile comprises at least about two, preferably at least six, and most preferably all of the genes or proteins selected from the group consisting of: VEGF, S100A3, PIGO, COL6A1, PSG1, F2RL1, MMP2, KIAA1539, MAP4K2, ITGB4 and CAPN1, an 11-marker gene signature. In alternate preferred embodiments, the genes/proteins are selected from one of the following 6-marker signatures: VEGF, S100A3, PIGO, COL6A1, PSG1 and F2RL1; or VEGF, MMP2, KIAA1539, MAP4K2, ITGB4 and CAPN1. The six-marker signatures are subsets of the 11-marker signature disclosed herein. All of these genes or proteins are upregulated in metastatic breast cancer patients that are likely to respond to treatment with a VEGF inhibitor.

In one aspect of the invention, the expression of genes or proteins in a tumor tissue and/or serum sample from a patient afflicted with metastatic breast cancer is assayed using array or immunohistochemistry techniques to identify the expression of the genes or proteins in the GPEPs consisting of: VEGF, S100A3, PIGO, COL6A1, PSG1 and F2RL1; or alternatively, VEGF, MMP2, KIAA1539, MAP4K2, ITGB4 and CAPN1. According to the invention, some or all of these genes/proteins are differentially expressed in metastatic breast cancer patients who are most likely to respond to VEGF-inhibitor therapy. Specifically, these genes/proteins were found to be up-regulated (over-expressed) in patients who are likely to respond positively to therapy with a VEGF inhibitor.

Methods of the present invention comprise (a) obtaining a biological sample (preferably a tumor tissue and/or serum sample) of a patient presenting with metastatic breast cancer; (b) contacting the sample with nucleic acid probes or antibodies specific for two or more members of a GPEP, PEP or GEP identified herein, and (c) determining whether two or more of the members of the profile are up-regulated (over-expressed).

The predictive value of the GPEPs for determining the likelihood of responsiveness to a VEGF inhibitor increases with the number of the members found to be up-regulated. Preferably, at least about two, more preferably at least about four, and most preferably about six, of the genes and/or proteins in the present GPEP are overexpressed. In a preferred embodiment of an assay in which tumor tissue is used as the biological sample, samples of normal (undiseased) margin tissue (tissue surrounding the lesion site) as well as other control tissues are assayed simultaneously, using the same reagents and under the same conditions, with the primary lesion site. In a preferred embodiment of an assay in which serum is used as the biological sample, serum samples from normal (non-cancer) patients and normal serum samples, to which known levels of VEGF protein have been added in order to provide a reference standard, are assayed simultaneously, using the same reagents and under the same conditions, with the patient's serum. Preferably, in both types as assays, expression levels of at least two reference proteins also are measured at the same time and under the same conditions to ensure that the assay is working properly. The assay is deemed to be working properly if the expression levels of the reference genes/proteins are substantially the same (not differtially expressed) in both the patient sample and the control samples.

In a currently preferred embodiment, the present invention comprises assays and methods for determining protein expression profiles that are indicative of the likelihood of responsiveness to therapy with a VEGF inhibitor in a metastatic breast cancer patient. In this embodiment, the present method comprises (a) obtaining a biological sample (tumor tissue or serum) of a patient afflicted with metastatic breast cancer; (b) contacting the sample with antibodies specific for the following proteins: VEGF, S100A3, PIGO, COL6A1, PSG1, F2RL1, MMP2, KIAA1539, MAP4K2, ITGB4 and CAPN1; or, alternatively, one of the following subsets: VEGF, S100A3, PIGO, COL6A1, PSG1 and F2RL1; or VEGF, MMP2, KIAA1539, MAP4K2, ITGB4 and CAPN1; and (c) determining whether two or more of the proteins are up-regulated (over-expressed) compared to normal (non-cancer) patients. The predictive value of the protein expression profile for determining the responsiveness of the patient to treatment with a VEGF inhibitor increases with the number of these proteins that are found to be up-regulated in accordance with the invention. Preferably, at least about two, more preferably at least about four, and most preferably about six, of the proteins in the present PEPs are upregulated in patients that are likely to respond to therapy with a VEGF inhibitor.

In another currently preferred embodiment, the present invention comprises gene expression profiles that are indicative of the likelihood of responsiveness to therapy with a VEGF inhibitor in a metastatic breast cancer patient. In this embodiment, the present method comprises (a) obtaining a biological sample (tumor tissue or serum) of a patient afflicted with metastatic breast cancer; (b) contacting the sample with nucleic acid probes specific for the following genes (e.g, DNA or mRNA): VEGF, S100A3, PIGO, COL6A1, PSG1, F2RL1, MMP2, KIAA1539, MAP4K2, ITGB4 and CAPN1; or, alternatively, one of the following subsets: VEGF, S100A3, PIGO, COL6A1, PSG1 and F2RL1; or VEGF, MMP2, KIAA1539, MAP4K2, ITGB4 and CAPN1; and (c) determining whether two or more of the members of the profile are up-regulated (over-expressed) compared to normal (non-cancer) patients. The predictive value of the gene expression profile for determining the responsiveness of the patient to treatment with a VEGF inhibitor increases with the number of these genes that are found to be up-regulated in accordance with the invention. Preferably, at least about two, more preferably at least about four, and most preferably about six, of the genes in the present GEPs are upregulated in patients that are likely to respond to therapy with a VEGF inhibitor.

The biological sample preferably is a sample of the patient's serum. Alternatively, the sample may be tumor tissue. Preferably, expression of at least two reference genes or proteins also is measured simultaneously with the measurement of the genes or proteins in the present GPEPs. The currently preferred reference genes are beta-actin (ACTB), glyceraldehyde-3-phosphate dehydrogenase (GAPDH), beta glucoronidase (GUSB), large ribosomal protein (RPLP0) and/or transferrin receptor (TRFC).

The present invention further comprises assays for determining the gene and/or protein expression profile in a patient's sample, and instructions for using the assay. The assay may be based on detection of nucleic acids (e.g., using nucleic acid probes specific for the nucleic acids of interest, preferably mRNA) or proteins or peptides (e.g., using nucleic acid probes or antibodies specific for the proteins/peptides of interest). In one embodiment, the assay comprises an immunohistochemistry (IHC) test in which test and control tissue samples, preferably arrayed in a tissue microarray (TMA), are contacted with antibodies specific for the proteins/peptides identified in the present PEP as being indicative of the likelihood that the patient's disease will respond to therapy with a VEGF inhibitor. In another preferred embodiment, the assay comprises an enzyme-linked immunosorbant assay (ELISA) in which serum samples, which preferably have been treated to release the proteins from circulating cells, are arrayed in a microtiter plate or other substrate and contacted with antibodies specific for the proteins/peptides identified in the present PEP as being indicative of the likelihood of responsiveness to treatment with a VEGF inhibitor.

Inclusion of any of the biomarker or diagnostic methods described herein as part of treatment and/or monitoring regimens to predict the effectiveness of treatment of a metastatic breast cancer patient with an anti-VEGF therapeutic provides an advantage over treatment or monitoring regimens that do not include such a biomarker or diagnostic step, in that only that patient population which needs or derives most benefit from such therapy need be treated. In particular, patients who are predicted not to benefit from treatment with a VEGF inhibitor (where responsiveness to the therapy is not predicted) can be treated with alternate therapies that are likely to be more effective for those patients.

The present invention further provides a method for treating a patient having metastatic breast cancer, comprising the step of determining a patient's likely responsiveness to treatment with a VEGF inhibitor using one or more of the present GPEP signatures to predict responsiveness; and a step of administering the patient an appropriate treatment regimen for metastatic breast cancer given the patient's age, gender, or other therapeutically relevant criteria.

Tables 2, 3 and 4 include the NCBI Accession No. of at least one variant of each gene. Other variants of these genes and proteins exist, which can be readily ascertained by reference to an appropriate database such as NCBI Entrez (available via the NIH website). Alternate names for the genes and proteins listed also can be determined from the NCBI site. All of the genes and/or proteins listed in Tables 2, 3 and 4 are up-regulated (overexpressed) in the tumor tissue (both primary tumor and metastatic tumor tissue) and blood or sera (circulating cells and circulating proteins in sera) of patients who are likely to respond to treatment with a VEGF inhibitor.

### DEFINITIONS

For convenience, the meaning of certain terms and phrases employed in the specification, examples, and appended claims are provided below. The definitions are not meant to be limiting in nature and serve to provide a clearer understanding of certain aspects of the present invention.

The term “genome” is intended to include the entire DNA complement of an organism, including the nuclear DNA component, chromosomal or extrachromosomal DNA, as well as the cytoplasmic domain (e.g., mitochondrial DNA).

The term “gene” refers to a nucleic acid sequence that comprises control and most often coding sequences necessary for producing a polypeptide or precursor. Genes, however, may not be translated and instead code for regulatory or structural RNA molecules.

A gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions. The term “gene” as used herein includes variants of the genes identified in Tables 2, 4 and 6.

The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

The terms “gene expression profile” or “GEP” or “gene signature” refer to a group of genes expressed by a particular cell or tissue type wherein presence of the genes or transcriptional products thereof, taken individually (as with a single gene marker) or together or the differential expression of such, is indicative/predictive of a certain condition.

The phrase “single-gene marker” or “single gene marker” refers to a single gene (including all variants of the gene) expressed by a particular cell or tissue type wherein presence of the gene or transcriptional products thereof, taken individually the differential expression of such, is indicative/predictive of a certain condition.

The phrase “gene-protein expression profile “GPEP” as used herein refers to the group of genes and proteins expressed by a particular cell or tissue type wherein presence of the genes and the proteins, taken together or the differential expression of such, is indicative/predictive of a certain condition. GPEPs are comprised of one or more sets of GEPs and PEPs.

The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like.

The term “complementary” as it relates to nucleic acids refers to hybridization or base pairing between nucleotides or nucleic acids, such as, for example, between the two strands of a double-stranded DNA molecule or between an oligonucleotide probe and a target are complementary.

As used herein, an “expression product” is a biomolecule, such as a protein or mRNA, which is produced when a gene in an organism is expressed. An expression product may comprise post-translational modifications. The polypeptide of a gene may be encoded by a full length coding sequence or by any portion of the coding sequence.

The terms “amino acid” and “amino acids” refer to all naturally occurring L-alpha-amino acids. The amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

The term “amino acid sequence variant” refers to molecules with some differences in their amino acid sequences as compared to a native sequence. The amino acid sequence variants may possess substitutions, deletions, and/or insertions at certain positions within the amino acid sequence. Ordinarily, variants will possess at least about 70% homology to a native sequence, and preferably, they will be at least about 80%, more preferably at least about 90% homologous to a native sequence.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions or deletions of amino acid residues that still maintain the properties of the parent polypeptide.

The term “derivative” is used synonymously with the term “variant” and refers to a molecule that has been modified or changed in any way relative to a reference molecule or starting molecule.

The term “respond” or “responsive” as used herein with respect to treatment with a VEGF inhibitor means a reduction in tumor burden or tumor size of at least thirty percent (30%) resulting from administration of the VEGF inhibitor.

A “VEGF inhibitor” is a therapeutic agent that blocks the growth of new blood vessels in the human body by blocking, inhibiting or reducing the activity of vascular endothelial growth factor (VEGF). VEGF is a signal protein produced by cells that stimulates the growth of new blood vessels. It is part of the system that restores the oxygen supply to tissues when blood circulation is inadequate. VEGF's normal function is to create new blood vessels during embryonic development, new blood vessels after injury, muscle following exercise, and new vessels (collateral circulation) to bypass blocked vessels. However, when VEGF is overexpressed, it can contribute to disease. Solid cancers cannot grow beyond a limited size without an adequate blood supply; cancers that can express VEGF are able to grow and metastasize. Drugs that can inhibit VEGF can help control or slow growth of such cancers. VEGF inhibitors currently available include monoclonal antibodies such as bevacizumab (Avastin®), antibody derivatives such as ranibizumab (Lucentis®), or orally-available small molecules that inhibit the tyrosine kinases stimulated by VEGF, including lapatinib (Tykerb®), sunitinib (Sutent®), sorafenib (Nexavar®), axitinib, and pazopanib.

The present invention contemplates several types of compositions, such as antibodies, which are amino acid based including variants and derivatives. These include substitutional, insertional, deletion and covalent variants and derivatives. As such, included within the scope of this invention are polypeptide based molecules containing substitutions, insertions and/or additions, deletions and covalently modifications. For example, sequence tags or amino acids, such as one or more lysines, can be added to the polypeptide sequences of the invention (e.g., at the N-terminal or C-terminal ends). Sequence tags can be used for polypeptide purification or localization. Lysines can be used to increase solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants,” when referring to proteins, are those with one or more amino acids in the native or starting amino acid sequence removed. Ordinarily, deletional variants will have one or more amino acids deleted in a particular region of the molecule.

“Covalent derivatives,” when referring to proteins, include modifications of a native or starting protein with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

Covalent derivatives specifically include fusion molecules in which proteins of the invention are covalently bonded to a non-proteinaceous polymer. The non-proteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The proteins may be linked to various non-proteinaceous polymers, such as polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337.

“Features” when referring to proteins are defined as distinct amino acid sequence-based components of a molecule. Features of the proteins of the present invention include surface manifestations, local conformational shape, folds, loops, half-loops, domains, half-domains, sites, termini or any combination thereof.

As used herein when referring to proteins the term “surface manifestation” refers to a polypeptide based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions).

As used herein when referring to proteins the term “half-domain” means portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid based embodiments is used synonymous with “amino acid residue” and “amino acid side chain”. A site represents a position within a peptide or polypeptide that may be modified, manipulated, altered, derivatized or varied within the polypeptide based molecules of the present invention.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH₂)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

A “protein” means a polymer of amino acid residues linked together by peptide bonds. The term, as used herein, refers to proteins, polypeptides, and peptides of any size, structure, or function. Typically, however, a protein will be at least 50 amino acids long. In some instances the protein encoded is smaller than about 50 amino acids. In this case, the polypeptide is termed a peptide. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues is an artificial chemical analogue of a corresponding naturally occurring amino acid.

The term “protein expression” refers to the process by which a nucleic acid sequence undergoes translation such that detectable levels of the amino acid sequence or protein are expressed.

The terms “protein expression profile” or “PEP” or “protein expression signature” refer to a group of proteins expressed by a particular cell or tissue type (e.g., neuron, coronary artery endothelium, or diseased tissue), wherein presence of the proteins taken individually (as with a single protein marker) or together or the differential expression of such proteins, is indicative/predictive of a certain condition.

The phrase “single-protein marker” or “single protein marker” refers to a single protein (including all variants of the protein) expressed by a particular cell or tissue type wherein presence of the protein or translational products of the gene encoding said protein, taken individually the differential expression of such, is indicative/predictive of a certain condition.

A “fragment of a protein,” as used herein, refers to a protein that is a portion of another protein. For example, fragments of proteins may comprise polypeptides obtained by digesting full-length protein isolated from cultured cells. In one embodiment, a protein fragment comprises at least about six amino acids. In another embodiment, the fragment comprises at least about ten amino acids. In yet another embodiment, the protein fragment comprises at least about sixteen amino acids.

The terms “array” and “microarray” refer to any type of regular arrangement of objects usually in rows and columns. As it relates to the study of gene and/or protein expression, arrays refer to an arrangement of probes (often oligonucleotide or protein based) or capture agents anchored to a surface which are used to capture or bind to a target of interest. Targets of interest may be genes, products of gene expression, and the like. The type of probe (nucleic acid or protein) represented on the array is dependent on the intended purpose of the array (e.g., to monitor expression of human genes or proteins). The oligonucleotide- or protein-capture agents on a given array may all belong to the same type, category, or group of genes or proteins. Genes or proteins may be considered to be of the same type if they share some common characteristics such as species of origin (e.g., human, mouse, rat); disease state (e.g., cancer); structure or functions (e.g., protein kinases, tumor suppressors); or same biological process (e.g., apoptosis, signal transduction, cell cycle regulation, proliferation, differentiation). For example, one array type may be a “cancer array” in which each of the array oligonucleotide- or protein-capture agents correspond to a gene or protein associated with a cancer. An “epithelial array” may be an array of oligonucleotide- or protein-capture agents corresponding to unique epithelial genes or proteins. Similarly, a “cell cycle array” may be an array type in which the oligonucleotide- or protein-capture agents correspond to unique genes or proteins associated with the cell cycle.

The terms “immunohistochemistry” or as abbreviated “IHC” as used herein refer to the process of detecting antigens (e.g., proteins) in a biologic sample by exploiting the binding properties of antibodies to antigens in said biologic sample.

The term “PCR” or “RT-PCR”, abbreviations for polymerase chain reaction technologies, as used here refer to techniques for the detection or determination of nucleic acid levels, whether synthetic or expressed.

The term “cell type” refers to a cell from a given source (e.g., a tissue, organ) or a cell in a given state of differentiation, or a cell associated with a given pathology or genetic makeup.

The term “activation” as used herein refers to any alteration of a signaling pathway or biological response including, for example, increases above basal levels, restoration to basal levels from an inhibited state, and stimulation of the pathway above basal levels.

The term “differential expression” refers to both quantitative as well as qualitative differences in the temporal and tissue expression patterns of a gene or a protein in diseased tissues or cells versus normal adjacent tissue. For example, a differentially expressed gene may have its expression activated or completely inactivated in normal versus disease conditions, or may be up-regulated (over-expressed) or down-regulated (under-expressed) in a disease condition versus a normal condition. Such a qualitatively regulated gene may exhibit an expression pattern within a given tissue or cell type that is detectable in either control or disease conditions, but is not detectable in both. Stated another way, a gene or protein is differentially expressed when expression of the gene or protein occurs at a higher or lower level in the diseased tissues or cells of a patient relative to the level of its expression in the normal (disease-free) tissues or cells of the patient and/or control tissues or cells.

The term “detectable” refers to an RNA expression pattern which is detectable via the standard techniques of polymerase chain reaction (PCR), reverse transcriptase-(RT) PCR, differential display, and Northern analyses, or any method which is well known to those of skill in the art. Similarly, protein expression patterns may be “detected” via standard techniques such as Western blots.

The term “complementary” as it relates to arrays refers to the topological compatibility or matching together of the interacting surfaces of a probe molecule and its target. The target and its probe can be described as complementary, and furthermore, the contact surface characteristics are complementary to each other.

The term “antibody” means an immunoglobulin, whether natural or partially or wholly synthetically produced. All derivatives thereof that maintain specific binding ability are also included in the term. The term also covers any protein having a binding domain that is homologous or largely homologous to an immunoglobulin binding domain. An antibody may be monoclonal or polyclonal. The antibody may be a member of any immunoglobulin class, including any of the human classes: IgG, IgM, IgA, IgD, and IgE.

The term “antibody fragment” refers to any derivative or portion of an antibody that is less than full-length. In one aspect, the antibody fragment retains at least a significant portion of the full-length antibody's specific binding ability, specifically, as a binding partner. Examples of antibody fragments include, but are not limited to, Fab, Fab′, F(ab′)2, scFv, Fv, dsFv diabody, and Fd fragments. The antibody fragment may be produced by any means. For example, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may comprise a single chain antibody fragment. In another embodiment, the fragment may comprise multiple chains that are linked together, for example, by disulfide linkages. The fragment may also comprise a multimolecular complex. A functional antibody fragment may typically comprise at least about 50 amino acids and more typically will comprise at least about 200 amino acids.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies. The preparation of antibodies, whether monoclonal or polyclonal, is know in the art. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

The term “biomarker” as used herein refers to a substance indicative of a biological state. According to the present invention, biomarkers include the GPEPs, PEPs, GEPs or combinations thereof. Biomarkers according to the present invention also include any compounds or compositions which are used to identify or signal the presence of one or more members of the GPEPs, PEPs, GEPs or combinations thereof disclosed herein. For example, an antibody created to bind to any of the proteins identified as a member of a PEP herein, may be considered useful as a biomarker, although the antibody itself is a secondary indicator.

The term “biological sample” or “biologic sample” refers to a sample obtained from an organism (e.g., a human patient) or from components (e.g., cells) of an organism. The sample may be of any biological tissue, organ, organ system or fluid. The sample may be a “clinical sample” which is a sample derived from a patient. Such samples include, but are not limited to, sputum, blood, blood cells (e.g., white cells), amniotic fluid, plasma, semen, bone marrow, and tissue or core or fine needle biopsy samples, urine, peritoneal fluid, and pleural fluid, or cells therefrom. Biological samples may also include sections of tissues such as frozen sections taken for histological purposes. A biological sample may also be referred to as a “patient sample.”

The term “condition” refers to the status of any cell, organ, organ system or organism. Conditions may reflect a disease state or simply the physiologic presentation or situation of an entity. Conditions may be characterized as phenotypic conditions such as the macroscopic presentation of a disease or genotypic conditions such as the underlying gene or protein expression profiles associated with the condition. Conditions may be benign or malignant.

The term “cancer” in an individual refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an individual, or may circulate in the blood stream as independent cells, such as leukemic cells.

The term “metastasis” or “metastatic” describes a cancer that has spread to other organs from the original tumor site, as well as the process by which cancer spreads from the place at which it first arose as a primary tumor to distant locations in the body.

The term “breast cancer” means a cancer of the breast tissue. “Metastatic breast cancer” is the most advanced stage (stage IV) of breast cancer. Cancer cells have spread past the breast and axillary (underarm) lymph nodes to other areas of the body where they continue to grow and multiply. Breast cancer has the potential to spread to almost any region of the body; the most common regions to which breast cancer spreads are the lymph nodes, chest wall, bone, lung, liver and brain.

The term “cell growth” is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

The term “tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

The term “lesion” or “lesion site” as used herein refers to any abnormal, generally localized, structural change in a bodily part or tissue. Calcifications or fibrocystic features are examples of lesions of the present invention.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient with cancer. The term “treatment” as used herein, unless otherwise indicated, refers to the act of treating.

The phrase “a method of treating” or its equivalent, when applied to, for example, cancer refers to a procedure or course of action that is designed to reduce, eliminate or prevent the number of cancer cells in an individual, or to alleviate the symptoms of a cancer. “A method of treating” cancer or another proliferative disorder does not necessarily mean that the cancer cells or other disorder will, in fact, be completely eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an individual, is nevertheless deemed an overall beneficial course of action.

The term “predicting” means a statement or claim that a particular event will occur in the future.

The term “prognosing” means a statement or claim that a particular biologic event will occur in the future.

The term “progression” or “cancer progression” means the advancement or worsening of or toward a disease or condition its characteristic presentation.

The term “therapeutically effective agent” means a composition that will elicit the biological or medical response of a tissue, organ, system, organism, animal or human that is being sought by the researcher, veterinarian, medical doctor or other clinician.

The term “therapeutically effective amount” or “effective amount” means the amount of the subject compound or combination that will elicit the biological or medical response of a tissue, organ, system, organism, animal or human that is being sought by the researcher, veterinarian, medical doctor or other clinician.

The term “correlate” or “correlation” as used herein refers to a relationship between two or more random variables or observed data values. A correlation may be statistical if, upon analysis by statistical means or tests, the relationship is found to satisfy the threshold of significance of the statistical test used.

### Determination of Gene Expression Profiles

Methods used to identify gene expression profiles indicative of whether a patient's condition is likely respond to treatment with a VEGF inhibitor are generally described here and further described in the Examples herein. Other methods for identifying gene and/or protein expression profiles are known; any of these alternative methods also could be used. See, e.g., Chen et al., NEJM, 356(1):11-20 (2007); Lu et al., PLOS Med., 3(12):e467 (2006); Wang et al., J. Clin. Oncol., 2299:1564 (2004); Golub et al., Science, 286:531-537 (1999).

In one method, parallel testing in which, in one track, those genes are identified which are over-/under-expressed as compared to normal (non-cancerous) tissue and/or disease tissue from patients that experienced different outcomes; and, in a second track, those genes are identified comprising chromosomal insertions or deletions as compared to the same normal and disease samples. These two tracks of analysis produce two sets of data. The data are analyzed and correlated using an algorithm which identifies the genes of the gene expression profile (i.e., those genes that are differentially expressed in the cancer tissue of interest). Positive and negative controls may be employed to normalize the results, including eliminating those genes and proteins that also are differentially expressed in normal tissues from the same patients, and is disease tissue having a different outcome, and confirming that the gene expression profile is unique to the cancer of interest.

As an initial step, biological samples are acquired from patients presenting with metastatic breast cancer (e.g., the metastases may occur in remaining breast tissue, including the breast unaffected by the primary tumor, or in the chest wall, bone, liver, lung, lymph nodes, brain or other location). The biological samples preferably include tissue samples and matched blood and/or serum samples from each patient. Tissue samples preferably include samples of the primary resected tumor, metastatic tumor tissue and normal (undiseased) marginal tissue from each patient. Clinical information associated with each sample, including treatment with chemotherapeutic drugs, surgery, radiation or other treatment, outcome of the treatments and recurrence or metastasis of the disease, is recorded in a database. Clinical information also includes information such as age, sex, medical history, treatment history, symptoms, family history, recurrence (yes/no), etc. Samples of normal (non-cancerous) tissue of different types (e.g., lung, brain, prostate) as well as samples of non-breast cancers (e.g., melanoma, breast cancer, ovarian cancer) can be used as positive controls. Samples of normal undiseased breast tissue from a set of healthy individuals can be used as positive controls, and breast tumor samples from patients whose cancer did recur/metastasize may be used as negative controls.

Gene expression profiles (GEPs) are then generated from the biological samples based on total RNA according to well-established methods. Briefly, a typical method involves isolating total RNA from the biological sample, amplifying the RNA, synthesizing cDNA, labeling the cDNA with a detectable label, hybridizing the cDNA with a genomic array, such as the Affymetrix U133A+B GeneChip®, and determining binding of the labeled cDNA with the genomic array by measuring the intensity of the signal from the detectable label bound to the array. See, e.g., the methods described in Lu, et al., Chen, et al. and Golub, et al., supra, and the references cited therein, which are incorporated herein by reference. The resulting expression data are input into a database.

mRNAs in the tissue samples or blood/serum samples can be analyzed using commercially available or customized probes or oligonucleotide arrays, such as cDNA or oligonucleotide arrays. The use of these arrays allows for the measurement of steady-state mRNA levels of thousands of genes simultaneously, thereby presenting a powerful tool for identifying effects such as the onset, arrest or modulation of uncontrolled cell proliferation. Hybridization and/or binding of the probes on the arrays to the nucleic acids of interest from the cells can be determined by detecting and/or measuring the location and intensity of the signal received from the labeled probe or used to detect a DNA/RNA sequence from the sample that hybridizes to a nucleic acid sequence at a known location on the microarray. The intensity of the signal is proportional to the quantity of cDNA or mRNA present in the sample tissue. Numerous arrays and techniques are available and useful. Methods for determining gene and/or protein expression in sample tissues are described, for example, in U.S. Pat. No. 6,271,002; U.S. Pat. No. 6,218,122; U.S. Pat. No. 6,218,114; and U.S. Pat. No. 6,004,755; and in Wang et al., J. Clin. Oncol., 22(9):1564-1671 (2004); Golub et al, (supra); and Schena et al., Science, 270:467-470 (1995); all of which are incorporated herein by reference.

The gene analysis aspect may interrogate gene expression as well as insertion/deletion data. As a first step, RNA is isolated from the tissue, blood or serum samples and labeled. Parallel processes are run on the sample to develop two sets of data: (1) over-/under-expression of genes based on mRNA levels; and (2) chromosomal insertion/deletion data. These two sets of data are then correlated by means of an algorithm. Over-/under-expression of the genes in each tissue sample are compared to gene expression in the normal (non-cancerous) samples and other control samples, and a subset of genes that are differentially expressed in the cancer tissue is identified. Preferably, levels of up- and down-regulation are distinguished based on fold changes of the intensity measurements of hybridized microarray probes. A difference of about 2.0 fold or greater is preferred for making such distinctions, or a p-value of less than about 0.05. That is, before a gene is said to be differentially expressed in diseased or suspected diseased versus normal cells, the diseased cell is found to yield at least about 2 times greater or less intensity of expression than the normal cells. Generally, the greater the fold difference (or the lower the p-value), the more preferred is the gene for use as a diagnostic or prognostic tool. Genes identified for the gene signatures of the present invention have expression levels that result in the generation of a signal that is distinguishable from those of the normal or non-modulated genes by an amount that exceeds background using clinical laboratory instrumentation.

Statistical values can be used to confidently distinguish modulated from non-modulated genes and noise. Statistical tests can identify the genes most significantly differentially expressed between diverse groups of samples. The Student's t-test is an example of a robust statistical test that can be used to find significant differences between two groups. The lower the p-value, the more compelling the evidence that the gene is showing a difference between the different groups. Nevertheless, since microarrays allow measurement of more than one gene at a time, tens of thousands of statistical tests may be run at one time. Because of this, it is unlikely to observe small p-values just by chance, and adjustments using a Sidak correction or similar step as well as a randomization/permutation experiment can be made. A p-value less than about 0.05 by the t-test is evidence that the expression level of the gene is significantly different. More compelling evidence is a p-value less than about 0.05 after the Sidak correction is factored in. For a large number of samples in each group, a p-value less than about 0.05 after the randomization/permutation test is the most compelling evidence of a significant difference.

Another parameter that can be used to select genes that generate a signal that is greater than that of the non-modulated gene or noise is the measurement of absolute signal difference. Preferably, the signal generated by the differentially expressed genes differs by at least about 20% from those of the normal or non-modulated gene (on an absolute basis). It is even more preferred that such genes produce expression patterns that are at least about 30% different than those of normal or non-modulated genes. For smaller subsets of genes evaluated, such as profiles containing less than 30, less than or about 20 or less than or about 10 genes, the expression patterns may be at least about 40% or at least about 50% different than those of normal or non-modulated genes.

Differential expression analyses can be performed using commercially available arrays, for example, Affymetrix U133A+B GeneChip® arrays (Affymetrix, Inc.). These arrays have probe sets for the whole human genome immobilized on the chip, and can be used to determine up- and down-regulation of genes in test samples. Other substrates having affixed thereon human genomic DNA or probes capable of detecting expression products, such as those available from Affymetrix, Agilent Technologies, Inc. or Illumina, Inc. also may be used. Currently preferred gene microarrays for use in the present invention include Affymetrix U133A+B GeneChip® arrays and Agilent Technologies genomic cDNA microarrays. Instruments and reagents for performing gene expression analysis are commercially available. See, e.g., Affymetrix GeneChip® System. The expression data obtained from the analysis then is input into the database.

For chromosomal insertion/deletion analyses, data for the genes of each sample as compared to samples of normal tissue is obtained. The insertion/deletion analysis is generated using an array-based comparative genomic hybridization (“CGH”). Array CGH measures copy-number variations at multiple loci simultaneously, providing an important tool for studying cancer and developmental disorders and for developing diagnostic and therapeutic targets. Microchips for performing array CGH are commercially available, e.g., from Agilent Technologies. The Agilent chip is a chromosomal array which shows the location of genes on the chromosomes and provides additional data for the gene signature. The insertion/deletion data once acquired from this testing is also input into the database.

The analyses are carried out on the same samples from the same patients to generate parallel data. The same chips and sample preparation are used to reduce variability.

The expression of certain genes known as “reference genes” “control genes” or “housekeeping genes” also is determined, preferably at the same time, as a means of ensuring the veracity of the expression profile. Reference genes are genes that are consistently expressed in many tissue types, including cancerous and normal tissues, and thus are useful to normalize gene expression profiles. See, e.g., Silvia et al., BMC Cancer, 6:200 (2006); Lee et al., Genome Research, 12(2):292-297 (2002); Zhang et al., BMC Mol. Biol., 6:4 (2005). Determining the expression of reference genes in parallel with the genes in the unique gene expression profile provides further assurance that the techniques used for determination of the gene expression profile are working properly. The expression data relating to the reference genes also is input into the database. In a currently preferred embodiment, the following genes are used as reference genes: beta-actin (ACTB), glyceraldehyde-3-phosphate dehydrogenase (GAPDH), beta glucoronidase (GUSB), large ribosomal protein (RPLP0) and/or transferrin receptor (TRFC).

### Data Correlation

The differential expression data and the insertion/deletion data in the database may be correlated with the clinical outcomes information associated with each tissue sample also in the database by means of an algorithm to determine a gene expression profile for determining or predicting progression as well as recurrence of disease and/or disease-related presentations. Various algorithms are available which are useful for correlating the data and identifying the predictive gene signatures. For example, algorithms such as those identified in Xu et al., A Smooth Response Surface Algorithm For Constructing A Gene Regulatory Network, Physiol. Genomics 11:11-20 (2002), the entirety of which is incorporated herein by reference, may be used for the practice of the embodiments disclosed herein.

Another method for identifying gene expression profiles is through the use of optimization algorithms such as the mean variance algorithm widely used in establishing stock portfolios. One such method is described in detail in the patent application US Patent Application Publication No. 2003/0194734. Essentially, the method calls for the establishment of a set of inputs expression as measured by intensity) that will optimize the return (signal that is generated) one receives for using it while minimizing the variability of the return. The algorithm described in Irizarry et al., Nucleic Acids Res., 31:e15 (2003) also may be used. One useful algorithm is the JMP Genomics algorithm available from JMP Software.

The process of selecting gene expression profiles also may include the application of heuristic rules. Such rules are formulated based on biology and an understanding of the technology used to produce clinical results, and then are applied to output from the optimization method. For example, the mean variance method of gene signature identification can be applied to microarray data for a number of genes differentially expressed in subjects with cancer. Output from the method would be an optimized set of genes that could include some genes that are expressed in peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Other cells, tissues or fluids may also be used for the evaluation of differentially expressed genes, proteins or peptides. Of course, the rule can be applied prior to the formation of the efficient frontier by, for example, applying the rule during data pre-selection.

Other heuristic rules can be applied that are not necessarily related to the biology in question. For example, one can apply a rule that only a certain percentage of the portfolio can be represented by a particular gene or group of genes. Commercially available software such as the Wagner software readily accommodates these types of heuristics (Wagner Associates Mean-Variance Optimization Application). This can be useful, for example, when factors other than accuracy and precision have an impact on the desirability of including one or more genes.

As an example, the algorithm may be used for comparing gene expression profiles for various genes (or portfolios) to ascribe prognoses. The expression profiles (whether at the RNA or protein level) of each of the genes comprising the portfolio are fixed in a medium such as a computer readable medium. This can take a number of forms. For example, a table can be established into which the range of signals (e.g., intensity measurements) indicative of disease is input. Actual patient data can then be compared to the values in the table to determine whether the patient samples are normal or diseased. In a more sophisticated embodiment, patterns of the expression signals (e.g., fluorescent intensity) are recorded digitally or graphically. The gene expression patterns from the gene portfolios used in conjunction with patient samples are then compared to the expression patterns. Pattern comparison software can then be used to determine whether the patient samples have a pattern indicative of recurrence of the disease. Of course, these comparisons can also be used to determine whether the patient is not likely to experience disease recurrence. The expression profiles of the samples are then compared to the profile of a control cell. If the sample expression patterns are consistent with the expression pattern for recurrence of cancer then (in the absence of countervailing medical considerations) the patient is treated as one would treat a relapse patient. If the sample expression patterns are consistent with the expression pattern from the normal/control cell then the patient is diagnosed negative for the cancer.

A method for analyzing the gene signatures of a patient to determine prognosis of cancer is through the use of a Cox hazard analysis program. The analysis may be conducted using S-Plus software (commercially available from Insightful Corporation). Using such methods, a gene expression profile is compared to that of a profile that confidently represents relapse (i.e., expression levels for the combination of genes in the profile is indicative of relapse). The Cox hazard model with the established threshold is used to compare the similarity of the two profiles (known relapse versus patient) and then determines whether the patient profile exceeds the threshold. If it does, then the patient is classified as one who will relapse and is accorded treatment such as adjuvant therapy. If the patient profile does not exceed the threshold then they are classified as a non-relapsing patient. Other analytical tools can also be used to answer the same question such as, linear discriminate analysis, logistic regression and neural network approaches. See, e.g., software available from JMP statistical software.

Numerous other well-known methods of pattern recognition are available. The following references provide some examples:

Weighted Voting: Golub, T R., Slonim, D K., Tamaya, P., Huard, C., Gaasenbeek, M., Mesirov, J P., Coller, H., Loh, L., Downing, J R., Caligiuri, M A., Bloomfield, C D., Lander, E S. Molecular classification of cancer: class discovery and class prediction by gene expression monitoring. Science 286:531-537, 1999.

Support Vector Machines: Su, A I., Welsh, J B., Sapinoso, L M., Kern, S G., Dimitrov, P., Lapp, H., Schultz, P G., Powell, S M., Moskaluk, C A., Frierson, H F. Jr., Hampton, G M. Molecular classification of human carcinomas by use of gene expression signatures. Cancer Research 61:7388-93, 2001. Ramaswamy, S., Tamayo, P., Rifkin, R., Mukherjee, S., Yeang, C H., Angelo, M., Ladd, C., Reich, M., Latulippe, E., Mesirov, J P., Poggio, T., Gerald, W., Loda, M., Lander, E S., Gould, T R. Multiclass cancer diagnosis using tumor gene expression signatures Proceedings of the National Academy of Sciences of the USA 98:15149-15154, 2001.

K-nearest Neighbors: Ramaswamy, S., Tamayo, P., Rifkin, R., Mukherjee, S., Yeang, C H., Angelo, M., Ladd, C., Reich, M., Latulippe, E., Mesirov, J P., Poggio, T., Gerald, W., Loda, M., Lander, E S., Gould, T R. Multiclass cancer diagnosis using tumor gene expression signatures Proceedings of the National Academy of Sciences of the USA 98:15149-15154, 2001.

Correlation Coefficients: van't Veer L J, Dai H, van de Vijver M J, He Y D, Hart A, Mao M, Peters H L, van der Kooy K, Marton M J, Witteveen A T, Schreiber G J, Kerkhoven R M, Roberts C, Linsley P S, Bernards R, Friend S H. Gene expression profiling predicts clinical outcome of breast cancer, Nature. 2002 Jan. 31; 415(6871):530-6.

The gene expression analysis identifies a gene expression profile (GEP) unique to the cancer samples, that is, those genes which are differentially expressed by the cancer cells. This GEP then is validated, for example, using real-time quantitative polymerase chain reaction (RT-qPCR), which may be carried out using commercially available instruments and reagents, such as those available from Applied Biosystems.

### Determination of Protein Expression Profiles

Not all genes expressed by a cell are translated into proteins, therefore, once a GEP has been identified, it may also be desirable to ascertain whether proteins corresponding to some or all of the differentially expressed genes in the GEP also are differentially expressed by the same cells or tissue. Therefore, protein expression profiles (PEPs) are generated from the same suspect tissue control tissues used to identify the GEPs. PEPs also are used to validate the GEP in other individuals, e.g., breast cancer patients.

The preferred method for generating PEPs according to the present invention is by immunohistochemistry (IHC) analysis or ELISA assay. In these methods antibodies specific for the proteins in the PEP are used to interrogate tissue/serum samples from individuals of interest. Other methods for identifying PEPs are known, e.g. in situ hybridization (ISH) using protein-specific nucleic acid probes. See, e.g., Hofer et al., Clin. Can. Res., 11(16):5722 (2005); Volm et al., Clin. Exp. Metas., 19(5):385 (2002). Any of these alternative methods also could be used.

For determining the PEPs, samples of suspect tissue, including metastatic tumor tissue and normal margin tissue, or blood/serum samples, are obtained from patients. These typically are the same samples used for identifying the GEP. The tissue samples as well as the positive and negative control samples are arrayed on tissue microarrays (TMAs) to enable simultaneous analysis. TMAs consist of substrates, such as glass slides, on which up to about 1000 separate tissue samples are assembled in array fashion to allow simultaneous histological analysis. The tissue samples may comprise tissue obtained from preserved biopsy samples, e.g., paraffin-embedded or frozen tissues. Techniques for making tissue microarrays are well-known in the art. See, e.g., Simon et al., BioTechniques, 36(1):98-105 (2004); Kallioniemi et al, WO 99/44062; Kononen et al., Nat. Med., 4:844-847 (1998). In one method, a hollow needle is used to remove tissue cores as small as 0.6 mm in diameter from regions of interest in paraffin embedded tissues. The “regions of interest” are those that have been identified by a pathologist as containing the desired diseased or normal tissue. These tissue cores are then inserted in a recipient paraffin block in a precisely spaced array pattern. Sections from this block are cut using a microtome, mounted on a microscope slide and then analyzed by standard histological analysis. Each microarray block can be cut into approximately 100 to approximately 500 sections, which can be subjected to independent tests.

TMAs for the breast progression array are prepared using three tissue samples from each patient: one of breast tumor tissue, one from a lymph node and one of normal (undiseased) margin breast tissue (i.e., undiseased breast tissue surrounding the primary tumor site). The tumor tissues on the breast progression array include both metastatic and normal (non-cancerous) lymph nodes. Control arrays are also prepared: a normal screening array containing normal tissue samples from healthy, cancer-free individuals is included as a negative control, and a cancer survey array including tumor tissues from cancer patients afflicted with cancers other than breast cancer, are used as a positive control.

Proteins in the tissue samples may be analyzed by interrogating the TMAs using protein-specific agents, such as antibodies or nucleic acid probes, such as oligonucleotides or aptamers. Antibodies are preferred for this purpose due to their specificity and availability. The antibodies may be monoclonal or polyclonal antibodies, antibody fragments, and/or various types of synthetic antibodies, including chimeric antibodies, or fragments thereof. Antibodies are commercially available from a number of sources (e.g., Abcam, Cell Signaling Technology or Santa Cruz Biotechnology), or may be generated using techniques well-known to those skilled in the art. The antibodies typically are equipped with detectable labels, such as enzymes, chromogens or quantum dots, which permit the antibodies to be detected. The antibodies may be conjugated or tagged directly with a detectable label, or indirectly with one member of a binding pair, of which the other member contains a detectable label. Detection systems for use with are described, for example, in the website of Ventana Medical Systems, Inc. Quantum dots are particularly useful as detectable labels. The use of quantum dots is described, for example, in the following references: Jaiswal et al., Nat. Biotechnol., 21:47-51 (2003); Chan et al., Curr. Opin. Biotechnol., 13:40-46 (2002); Chan et al., Science, 281:435-446 (1998).

The use of antibodies to identify proteins of interest in the cells of a tissue, referred to as immunohistochemistry (IHC), is well established. See, e.g., Simon et al., BioTechniques, 36(1):98 (2004); Haedicke et al., BioTechniques, 35(1):164 (2003), which are hereby incorporated by reference. The IHC assay can be automated using commercially available instruments, such as the Benchmark instruments available from Ventana Medical Systems, Inc.

In one embodiment, the TMAs are contacted with antibodies specific for the proteins encoded by the genes identified in the gene expression study as being differentially expressed in breast cancer patients whose conditions had progressed to breast cancer in order to determine expression of these proteins in each type of tissue. The antibodies used to interrogate the TMAs are selected based on the genes having the highest level of differential expression. See data in Examples.

Proteins in the blood or serum samples may be analyzed by interrogating the whole blood, serum or plasma samples using protein-specific agents, such as antibodies or nucleic acid probes, such as oligonucleotides or aptamers. Determining differential protein expression from matched blood/serum samples may be performed in addition to, or as an alternative to, the IHC methods described herein in which tissue samples are analyzed. Methods for determining the presence and/or amounts of proteins in blood or serum are well-known.

The currently preferred method for determining protein expression is by immunoassay techniques. Any type of immunoassay format may be used, including, without limitation, enzyme immunoassays (EIA, ELISA), radioimmunoassay (RIA), fluoroimmunoassay (FIA), chemiluminescent immunoassay (CLIA), counting immunoassay (CIA), immunohistochemistry (IHC), agglutination, nephelometry, turbidimetry or Western Blot. These and other types of immunoassays are well-known and are described in the literature, for example, in Immunochemistry, Van Oss and Van Regenmortel (Eds), CRC Press, 1994; The Immunoassay Handbook, D. Wild (Ed.), Elsevier Ltd., 2005; and the references disclosed therein. In a preferred embodiment, of the present invention, an ELISA assay is used. See, e.g., Al-Moundhri et al., World J. Gastroenterol., 14(24):3879-83 (2008).

The results of the IHC, ELISA or other assay show that in individuals who are responsive to treatment with a VEGF inhibitor, the following proteins were up-regulated: VEGF, S100A3, PIGO, COL6A1, PSG1, F2RL1, MMP2, KIAA1539, MAP4K2, ITGB4 and CAPN1. Furthermore, two six-gene PEPs were identified and include the following proteins: VEGF, S100A3, PIGO, COL6A1, PSG1 and F2RL1; or VEGF, MMP2, KIAA1539, MAP4K2, ITGB4 and CAPN1. These proteins are upregulated in patients that were responsive to VEGF-inhibitor therapy compared with expression of these proteins in the serum/tissue samples from those patients who were not responsive to the therapy.

### Assays

The present invention further comprises methods and assays for determining or predicting whether a patient's condition is likely to progress to cancer. According to one aspect, a formatted IHC assay can be used for determining if a tissue sample exhibits any of the present GEPs, PEPs or GPEPs. In another aspect, a formatted ELISA assay can be used for determining if a serum sample exhibits any of the present GEPs, PEPs or GPEPs. The assays may be formulated into kits that include all or some of the materials needed to conduct the analysis, including reagents (antibodies, detectable labels, etc.) and instructions.

Any of the compositions described herein may be comprised in a kit. In a non-limiting example, reagents for the detection of PEPs, GEPs, or GPEPs are included in a kit. In one embodiment, antibodies to one or more of the expression products of the genes of the GPEPs disclosed herein are included. Antibodies may be included to provide concentrations of from about 0.1 μg/mL to about 500 μg/mL, from about 0.1 μg/mL to about 50 μg/mL or from about 1 μg/mL to about 5 μg/mL or any value within the stated ranges. The kit may further include reagents or instructions for creating or synthesizing further probes, labels or capture agents. It may also include one or more buffers, such as a nuclease buffer, transcription buffer, or a hybridization buffer, compounds for preparing a DNA template, cDNA, primers, probes or label, and components for isolating any of the foregoing. Other kits of the invention may include components for making a nucleic acid or peptide array including all reagents, buffers and the like and thus, may include, for example, a solid support.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. However, various combinations of components may be comprised in a vial or similar container. The kits of the present invention also will typically include a means for containing the detection reagents, e.g., nucleic acids or proteins or antibodies, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the invention. The dye may then be resuspended in any suitable solvent, such as DMSO.

Kits may also include components that preserve or maintain the compositions that protect against their degradation. Such kits generally will comprise, in suitable means, distinct containers for each individual reagent or solution.

The assay method of the invention comprises contacting a tissue sample from an individual with a group of antibodies specific for some or all of the genes or proteins in the present GPEP, and determining the occurrence of up- or down-regulation of these genes or proteins in the sample. The use of TMAs allows numerous samples, including control samples, to be assayed simultaneously.

The method preferably also includes detecting and/or quantitating control or “reference proteins”. Detecting and/or quantitating the reference proteins in the samples normalizes the results and thus provides further assurance that the assay is working properly. In a currently preferred embodiment, antibodies specific for one or more of the following reference proteins are included: beta-actin (ACTB), glyceraldehyde-3-phosphate dehydrogenase (GAPDH), beta glucoronidase (GUSB), large ribosomal protein (RPLP0) and/or transferrin receptor (TRFC).

In one embodiment, the assay and method comprises determining expression only of the overexpressed genes or proteins in the present GPEP. The method comprises obtaining a tissue sample from the patient, determining the gene and/or protein expression profile of the sample, and determining from the gene or protein expression profile whether at least one, more preferably at least two and most preferably at least six of the genes selected from the group consisting of VEGF, S100A3, PIGO, COL6A1, PSG1, F2RL1, MMP2, KIAA1539, MAP4K2, ITGB4 and CAPN1.

In one embodiment, the assay and method comprises determining expression of six overexpressed genes or proteins in the GPEP consisting of the genes: VEGF, S100A3, PIGO, COL6A1, PSG1 and F2RL1; or VEGF, MMP2, KIAA1539, MAP4K2, ITGB4 and CAPN1. The method preferably includes at least one reference protein, which may be selected from beta-actin (ACTB), glyceraldehyde-3-phosphate dehydrogenase (GAPDH), beta glucoronidase (GUSB), large ribosomal protein (RPLP0) and/or transferrin receptor (TRFC).

The present invention further comprises a kit containing reagents for conducting an IHC analysis of tissue samples or cells from individuals, e.g., patients, including antibodies specific for at least about two of the proteins in the GPEP and for any reference proteins. The antibodies are preferably tagged with means for detecting the binding of the antibodies to the proteins of interest, e.g., detectable labels. Preferred detectable labels include fluorescent compounds or quantum dots, however other types of detectable labels may be used. Detectable labels for antibodies are commercially available, e.g. from Ventana Medical Systems, Inc.

Immunohistochemical methods for detecting and quantitating protein expression in tissue samples are well known. Any method that permits the determination of expression of several different proteins can be used. See. e.g., Signoretti et al., “Her-2-neu Expression and Progression Toward Androgen Independence in Human Prostate Cancer,” J. Natl. Cancer Instit., 92(23):1918-25 (2000); Gu et al., “Prostate stem cell antigen (PSCA) expression increases with high gleason score, advanced stage and bone metastasis in prostate cancer,” Oncogene, 19:1288-96 (2000). Such methods can be efficiently carried out using automated instruments designed for immunohistochemical (IHC) analysis. Instruments for rapidly performing such assays are commercially available, e.g., from Ventana Molecular Discovery Systems or Lab Vision Corporation. Methods according to the present invention using such instruments are carried out according to the manufacturer's instructions.

Protein-specific antibodies for use in such methods or assays are readily available or can be prepared using well-established techniques. Antibodies specific for the proteins in the GPEP disclosed herein can be obtained, for example, from Cell Signaling Technology, Inc, Santa Cruz Biotechnology, Inc. or Abcam.

The present invention is illustrated further by the following non-limiting Examples.

Unless otherwise defined, all technical and scientific terms used herein have the same meaning as commonly understood by one of ordinary skill in the art to which this invention belongs. Although methods and materials similar or equivalent to those described herein can be used in the practice or testing of methods featured in the invention, suitable methods and materials are described below.

### EXAMPLES

### Example 1

### Gene Expression Profile (GEP) Analysis

Gene expression profiles of tumor biopsies were generated for 783 patients clinical studies CA NU2000 and CA NU3000. Metrics associated with the two clinical study subsets are shown in Table 1. The setting for both studies was inpatient treatment for metastatic breast cancer.

Gene expression data from the two studies was obtained via gene array methodology utilizing the Affymetrix HU133A-B GeneChip® whereby biopsy tissue samples were obtained from metastatic breast cancer patients who had been treated with bevacizumab and control samples. Among these patients were 684 patents that had responded to treatment with bevacizumab, and 99 that had not responded to the treatment. Response was determined as a reduction of ≧30% of tumor burden/size. Gene expression profiles (GEPs) then were generated from the biological samples based on total RNA according to well-established methods (See Affymetrix GeneChip® expression analysis technical manual, Affymetrix, Inc, Santa Clara, Calif.). Briefly, total RNA was isolated from the biological sample, amplified and cDNA synthesized. cDNA was then labeled with a detectable label, hybridized with a the Affymetrix HU133A-B GeneChip® genomic array, and binding of the cDNA to the array was quantified by measuring the intensity of the signal from the detectable cDNA label bound to the array.

The data were normalized together by Robust Microarray Analysis (RMA). The adenocarcinoma measure used for all analyses was pathological Cancer (pCR) in breast tissue based on central review of biopsies within 12 months of the initial mammography.

TABLE 1
Comparison of two clinical study subsets

| | Study Identifier | Study Identifier |
|---|---|---|
| | (CA NU2000) | (CA NU3000) |
| Drug treatment | Bevacizumab | Bevacizumab |
| Number of patients: | 356 | 427 |
| Total | | |
| Post-treatment tumor | Core needle | Core needle |
| biopsy type | | |
| Number of patients with | 298 | 386 |
| response to | | |
| bevacizumab: | | |
| Gene array type | Affymetrix | Affymetrix |
| | HU133A-B | HU133A-B |

As shown in the table, biopsy samples from 783 patients presenting with metastatic breast cancer that had been treated with bevacizumab were analyzed for gene expression. Of these, 684 of the patients had responded to treatment with the drug (responders), and 99 did not (non-responders). The gene expression data from the responders and the non-responders were analyzed to identify differences in gene expression between those patients that responded to bevacizumab treatment and those that did not respond.

Gene Ontology (GO) analysis was used as described by Lee H K et al 2005 (Tool for functional analysis of gene expression data sets. BMC Bioinformatics. 6: 269; See also: The Gene Ontology Consortium. “Gene ontology: tool for the unification of biology.” Nat. Genet. May 2000; 25(1):25-9 at http://www.geneontology.org) with 10,000 iterations of the Gene Score Re-sampling Algorithm. A gene network was built using the GeneGo program. Initial analyses used all detection of carcinomas. Subsequent analyses used the calcification subsets only.

To develop a predictive GPEP (gene-protein expression profile), 22,215 probe sets were filtered by removing (a) probe sets with low expression over all samples; and (b) probe sets with low variance over all samples. This yielded 11,318 probe sets for subsequent analyses. Normalized log 2(intensity) values were centered by subtracting the study-specific mean for each probe set, and rescaled by dividing by the pooled within-study standard deviation for each probe set.

A two-stage model-building approach was used to arrive at the best predictive model.

### Multi-Gene Markers

A fit was examined with multi-probe-set predictive models. Here, the pre-selected probe sets from the single-probe-set analyses were used as the starting point. Then the initial predictive models to each study were fit separately using a threshold gradient descent (TGD) method for regularized classification. Recursive feature elimination (RFE) was applied to attempt to simplify the models without appreciable loss of predictive accuracy.

The model selection criterion was the mean area under the ROC curve (AUC) from 50 replicates of a 4-fold cross-validation. Then from each RFE model series, here, one per study, the model with maximum difference between the selection criteria for the two studies was selected. The TGD method also was used to build predictive models based on expression of two individual probe sets.

Following the procedures outlined above, Signal-to-Noise ratios (S2N) were generated by comparing responders to bevacizumab treatment to non-responders in both trials (the whole data set).

S2N was calculated based upon the following formula:

S2N=|x₁−x₂|/(s₁+s₂)

where xᵢ is the mean for trial i and sᵢ is the standard deviation for trial i, i=1,2.

Genes with the 11 largest signal-to-noise (S2N) scores among those with a range of at least 2.5 for log 2 (expression intensity) and P-value <0.01 for a t-test of the mean expression difference between responders and non-responders are shown in Table 2. Gene and Protein Reference Sequences refers to the sequence identifier of the gene from the NCBI database.

TABLE 2
Genes having statistically significant signal-to-noise scores

| Gene | | Gene Reference | Signal to Noise | | SEQ ID |
|---|---|---|---|---|---|
| Symbol | Gene Name | Sequences* | score (S/N) | P value | NO |
| VEGF | Vascular endothelial | NM_001025366.2 | 0.625 | 0.00024 | 1 |
| | growth factor A | | | | |
| S100A3 | S100 calcium binding | NM_002960.1 | 0.851 | 0.00036 | 2 |
| | protein A3 | | | | |
| PIGO | Phosphatidylinositol | NM_032634.3 | 0.637 | 0.00091 | 3 |
| | glycan anchor | | | | |
| | biosynthesis, class O | | | | |
| COL6A1 | Collagen, type 4, alpha 1 | NM_001848.2 | 0.624 | 0.00045 | 4 |
| PSG1 | Pregnancy specific beta- | NM_006905.2 | 0.672 | 0.00062 | 5 |
| | 1 glycoprotein 1 | | | | |
| F2RL1 | Coagulation factor II | NM_005242.4 | 0.741 | 0.00027 | 6 |
| | (thrombin) receptor-like | | | | |
| | 1 | | | | |
| MMP2 | Matrix metallopeptidase | NM_004530.4 | 0.594 | 0.00026 | 7 |
| | 2 (gelatinase A, 72 kDa | | | | |
| | gelatinase, 72 kDa type- | | | | |
| | IV collagenase) | | | | |
| KIAA1539 | KIAA1539; Homo | NM_025182.2 | 0.535 | 0.00038 | 8 |
| | sapiens family with | | | | |
| | sequence similarity 214, | | | | |
| | member B (FAM214B) | | | | |
| MAP4K2 | Mitogen-activated | NM_004579.2 | 0.711 | 0.00025 | 9 |
| | protein kinase kinase | | | | |
| | kinase kinase 2 | | | | |
| ITGB4 | Integrin, beta 4 | NM_000213.3 | 0.632 | 0.00041 | 10 |
| CAPN1 | Captain 1, (mu/l) large | NM_001198868.1 | 0.698 | 0.00048 | 11 |
| | subunit | | | | |
| VEGF | Vascular endothelial | NM_003377.3 | | | 12 |
| | growth factor B | | | | |

*Gene sequence reference sequences have the “NM” prefix.

Table 2 sets forth an 11-gene profile or signature that is indicative of expression differences between responders and non-responders to treatment with bevacizumab among metastatic breast cancer patients who were treated with the drug. (For purposes of this invention, VEGF A and B are treated as one gene). This 11-gene GEP shows the top eleven differentially expressed genes in the pooled group of metastatic breast cancer patients treated with bevacizumab. All of the genes in the GEP were upregulated in the patients who were responders. The longest isoform of each gene is represented in Table 2; however, it is understood that other variants or isoforms of each gene may exist and that these are included within the embodiment of the gene.

Results of the analyses revealed the genes listed in Table 2 were identified as having the largest S2N scores and a relatively wide expression range.

Given these findings, the present invention contemplates the use of at least two, at least 4 or at least 6 of the genes as a gene expression profile, the differential expression of which, either alone or in conjunction with imaging, will serve as a predictor of cancer progression in individuals presenting with lesions of the breast tissue.

### Example 2

### Identification of GEP Subsets

The results of the analysis also identified two six-gene subsets that are indicative of the responsiveness of metastatic breast cancer patients to treatment with bevacizumab. These two six-gene GEPs are shown in Tables 3 and 4 respectively.

TABLE 3
Genes having statistically significant signal-to-noise scores (VEGF 1)

| Gene | | Gene Reference | Signal to Noise | | SEQ ID |
|---|---|---|---|---|---|
| Symbol | Gene Name | Sequences* | score (S/N) | P value | NO |
| VEGF | Vascular endothelial growth | NM_001025366.1 | 0.625 | 0.00024 | 1 |
| | factor A | | | | |
| | Vascular endothelial growth | NM_003377.3 | | | 12 |
| | factor B | | | | |
| S100A3 | S100 calcium binding protein | NM_002960.1 | 0.851 | 0.00036 | 2 |
| | A3 | | | | |
| PIGO | Phosphatidylinositol glycan | NM_032634.3 | 0.637 | 0.00091 | 3 |
| | anchor biosynthesis, class O | | | | |
| COL6A1 | Collagen, type 4, alpha 1 | NM_001848.2 | 0.624 | 0.00045 | 4 |
| PSG1 | Pregnancy specific beta-1 | NM_006905.2 | 0.672 | 0.00062 | 5 |
| | glycoprotein 1 | | | | |
| F2RL1 | Coagulation factor II | NM_005242.4 | 0.741 | 0.00027 | 6 |
| | (thrombin) receptor-like 1 | | | | |

TABLE 4
Genes having statistically significant signal-to-noise scores (VEGF 2)

| Gene | | Gene Reference | Signal to Noise | | SEQ ID |
|---|---|---|---|---|---|
| Symbol | Gene Name | Sequences* | score (S/N) | P value | NO |
| VEGF | Vascular endothelial growth | NM_001025366.1 | 0.625 | 0.00024 | 1 |
| | factor A | | | | |
| | Vascular endothelial growth | NM_003377.3 | | | 12 |
| | factor B | | | | |
| MMP2 | Matrix metallopeptidase 2 | NM_004530.4 | 0.594 | 0.00026 | 7 |
| | (gelatinase A, 72 kDa | | | | |
| | gelatinase, 72 kDa type-IV | | | | |
| | collagenase) | | | | |
| KIAA1539 | KIAA1539; Homo sapiens | NM_025182.2 | 0.535 | 0.00038 | 8 |
| | family with sequence | | | | |
| | similarity 214, member B | | | | |
| | (FAM214B) | | | | |
| MAP4K2 | Mitogen-activated protein | NM_004579.2 | 0.711 | 0.00025 | 9 |
| | kinase kinase kinase kinase 2 | | | | |
| ITGB4 | Integrin, beta 4 | NM_000213.3 | 0.632 | 0.00041 | 10 |
| CAPN1 | Captain 1, (mu/l) large subunit | NM_001198868.1 | 0.698 | 0.00048 | 11 |

The results of the analyses using the two 6-gene subsets are shown in Table 5. These data illustrate that the six-marker model for both subsets (the presence of increased expression of these genes) predicted responsiveness to treatment with bevacizumab with an accuracy of almost 90% for signature 1 and 80% for signature 2 from initial presentations of either calcifications or fibrocystic changes, respectively, in the tissue.

TABLE 5
Six-marker GEPs are predictive of therapeutic response

| | | Study Identifier | | | Study Identifier | | |
|---|---|---|---|---|---|---|---|
| | | (CA NU2000) | | | (CA NU3000) | | |
| | | | | Detec- | | | Detec- |
| | | | | tion | | | tion |
| Model | Subset | R | N | Rate | R | N | Rate |
| VEGF 1 | All | 298 | 356 | 0.84 | 386 | 427 | 0.90 |
| and 2 | patients | | | | | | |
| combined | | | | | | | |
| (11-gene | | | | | | | |
| GPEP) | | | | | | | |
| VEGF 1 | Predicted | 256 | 288 | 0.88 | 296 | 335 | 0.88 |
| | response | | | | | | |
| | cancer | | | | | | |
| VEGF 2 | Predicted | 226 | 280 | 0.780 | 231 | 293 | 0.78 |
| | response | | | | | | |

R = True number of detections, N = Total number of patients in subset, Detection Rate = R/N. The detection rate for each condition for all patients, and for only patients with estimated detection probability was set at an arbitrary threshold of 0.5 based on expression level.

Consequently, the studies provide six-marker GEPs where the level of expression may be employed as a tool, either alone or in conjunction with other GEPs or imaging techniques, to predict progression to cancer or responsiveness to a therapeutic such as a VEGF inhibitor.

### Example 3

### Immunohistochemistry Analysis

Immunohistochemistry (IHC) analysis was used to confirm that expression of the proteins corresponding to the genes in the GEPs of the invention also is upregulated in patients who were responders to bevacizumab therapy.

### Tissue Microarrays

Tissue samples were obtained from post-treatment tumor biopsies of 783 patients presenting with metastatic breast cancer (356 patients in clinical study CA NU2000, and 427 in clinical study CA NU3000). Matched serum samples also were obtained from all patients. All patients had been treated with bevacizumab (Avastin®) for the metastases. Of these, 298 patients from CA NU2000 and 386 patients from CA NU3000 (684 patients total) evidenced a positive response to bevacizumab, as determined by an at least thirty percent (30%) reduction in tumor burden/size.

In this study, formalin fixed paraffin embedded breast cancer metastases specimens from the metastatic breast cancer patients were evaluated for tumor size, histologic grade and status. Using the techniques described above, a Gene Expression Profile (GEP) was generated from these specimens and comprised genes which were found to be differentially expressed in patients whose metastatic breast cancer had responded positively to treatment with bevacizumab compared to patients whose disease did not respond to the treatment. The following genes comprised the 11-gene GEP present in the responders: VEGF, S100A3, PIGO, COL6A1, PSG1, F2RL1, MMP2, KIAA1539, MAP4K2, ITGB4 and CAPN1.

Further, two six-gene GEPs of differentially expressed genes comprising subsets of the above 11-gene GEPs were identified in the pooled groups of patients that were responders to bevacizumab. The two subsets were: VEGF, S100A3, PIGO, COL6A1, PSG1 and F2RL1; and VEGF, MMP2, KIAA1539, MAP4K2, ITGB4 and CAPN1.

### Tissue Microarrays (TMAs)

Tissue microarrays were prepared using the tumor biopsies from the primary and metastatic tumors of the patients in CA NU2000 and CA NU3000, as well as normal (non-cancerous) breast tissue from patients described above. TMAs also were prepared containing control tissue samples from non-breast cancers; the control tissues are included to confirm that the GPEP is unique to breast cancer. A test array containing normal non-cancerous tissues was included as a control for antibody dilution, and also as another negative control. The TMAs used in this study are described in Table A.

TABLE A
Tissue MicroArrays

| | |
|---|---|
| Metastatic | This array contained the patient samples obtained from patients |
| Breast Cancer | afflicted with metastatic breast cancer that had been treated with |
| Array | bevacizumab. The samples include tumor tissue from the primary |
| | breast tumor, metastatic tumor(s) (breast, liver, lung and/or bone), |
| | lymph nodes and normal (undiseased) breast tissue samples from |
| | each patient. |
| Normal | This array contained samples of normal (non-cancerous) tissue. |
| Screening | The normal tissues in this array include lung, breast, ovarian, |
| Array | placenta, brain, pancreas, parotid gland, skin, breast, prostate and |
| | lymph node. This array was included as a negative control to |
| | confirm that the GPEP is unique to non-recurrent breast cancer |
| | tissue, i.e., that it does not occur in any normal tissues. |
| Cancer | This array contained tumor samples for cancers including lung |
| Screening | adeno, breast adeno, ovarian adeno, brain cancer (normal and |
| Survey Array | glio), pancreas adeno, parotid gland cancer, melanoma, skin |
| | cancer, breast cancer and prostate adeno. This array was included |
| | as a negative control to confirm that the GPEP is unique to non- |
| | recurrent breast cancer tissue, i.e., that it does not occur in any |
| | other cancer tissues. |
| Test Array | This array contained samples of the following normal (non- |
| (TE-30 Array) | cancerous) tissues: breast, liver, lung, prostate and breast. This |
| | array is included for antibody dilution and as a negative control to |
| | confirm that the GPEP is unique to non-recurrent breast cancer |
| | tissue, i.e., that it does not occur in any of these normal tissues. |

### TMA Protocol

Tissue cores from donor block containing the patient tissue samples were inserted into a recipient paraffin block. These tissue cores are punched with a thin walled, sharpened borer. An X-Y precision guide allowed the orderly placement of these tissue samples in an array format.

Presentation: TMA sections were cut at 4 microns and are mounted on positively charged glass microslides. Individual elements were 0.6 mm in diameter, spaced 0.2 mm apart.

Elements: In addition to TMAs containing the recurrent and non-recurrent breast cancer samples, screening arrays were produced made up of cancer tissue samples other than recurrent breast cancer, 2 each from a different patient. Additional normal tissue samples were included for quality control purposes.

The TMAs were designed for use with the specialty staining and immunohistochemical methods described below for gene expression screening purposes, by using monoclonal and polyclonal antibodies over a wide range of characterized tissue types. Accompanying each array was an array locator map and spreadsheet containing patient diagnostic, histologic and demographic data for each element.

### Immunohistochemical Staining

Immunohistochemical staining techniques were used for the visualization of tissue (cell) proteins present in the tissue samples. These techniques were based on the immunoreactivity of antibodies and the chemical properties of enzymes or enzyme complexes, which react with colorless substrate-chromogens to produce a colored end product. Initial immunoenzymatic stains utilized the direct method, which conjugated directly to an antibody with known antigenic specificity (primary antibody).

A modified labeled avidin-biotin technique was employed in which a biotinylated secondary antibody formed a complex with peroxidase-conjugated streptavidin molecules. Endogenous peroxidase activity was quenched by the addition of 3% hydrogen peroxide. The specimens then were incubated with the primary antibodies followed by sequential incubations with the biotinylated secondary link antibody (containing anti-rabbit or anti-mouse immunoglobulins) and peroxidase labeled streptavidin. The primary antibody, secondary antibody, and avidin enzyme complex is then visualized utilizing a substrate-chromogen that produces a brown pigment at the antigen site that is visible by light microscopy.

VEGF and MMP2 antibodies were obtained from Cell Signaling Technology (Danvers, Mass.) (include cat. nos.). COL6A1, F2RL1, MAP4K2, ITGB4 and CAPN1 antibodies are available from LifeSpan Biosciences (Seattle, Wash.). S100A3, PIGO and KIAA1539 antibodies are available from Abcam (Cambridge, Mass.).

### Automated Immunohistochemistry Staining Procedure (IHC):

1. Heat-induced epitope retrieval (HIER) using 10 mM Citrate buffer solution, pH 6.0, was performed as follows:
   a. Deparaffinized and rehydrated sections were placed in a slide staining rack.
   b. The rack was placed in a microwaveable pressure cooker; 750 ml of 10 mM Citrate buffer pH 6.0 was added to cover the slides.
   c. The covered pressure cooker was placed in the microwave on high power for 15 minutes.
   d. The pressure cooker was removed from the microwave and cooled until the pressure indicator dropped and the cover could be safely removed.
   e. The slides were allowed to cool to room temperature, and immunohistochemical staining was carried out.
2. Slides were treated with 3% H₂O₂ for 10 min. at RT to quench endogenous peroxidase activity.
3. Slides were rinsed gently with phosphate buffered saline (PBS).
4. The primary antibodies were applied at the predetermined dilution (according to Cell Signaling Technology's Specifications) for 30 min at room temperature. Normal mouse or rabbit serum 1:750 dilution was applied to negative control slides.
5. Slides were rinsed with phosphate buffered saline (PBS).
6. Secondary biotinylated link antibodies* were applied for 30 min at room temperature.
7. Slides were rinsed with phosphate buffered saline (PBS).
8. The slides were treated with streptavidin-HRP (streptavidin conjugated to horseradish peroxidase)** for 30 min at room temperature.
9. Slides were rinsed with phosphate buffered saline (PBS).
10. The slides were treated with substrate/chromogen*** for 10 min at room temperature.
11. Slides were raised with distilled water.
12. Counter stain in Hematoxylin was applied for 1 min.
13. Slides were washed in running water for 2 min.
14. The slides were then dehydrated, cleared and the cover glass was mounted
   *Secondary antibody: biotinylated anti-chicken and anti-mouse immunoglobulins in phosphate buffered saline (PBS), containing carrier protein and 15 mM sodium azide.
   **Streptavidin-HRP in PBS containing carrier protein and anti-microbial agents from Ventana,
   ***Substrate-Chromogen is substrate-imidazole-HCl buffer pH 7.5 containing H2O2 and anti-microbial agents, DAB-3,3′-diaminobenzidine in chromogen solution from Ventana.

All primary antibodies were titrated to dilutions according to manufacturer's specifications. Staining of TE30 Test Array slides (described above) was performed with and without epitope retrieval (HIER). The slides were screened by a pathologist to determine the optimal working dilution. Pretreatment with HIER provided strong specific staining with little to no background. The above immunohistochemical staining was carried out using a Benchmark instrument from Ventana Medical Systems, Inc.

### Scoring Criteria

Staining was scored on a 0-3+ scale, with 0=no staining, and trace (tr) being less than 1+ but greater than 0. The scoring procedures are described in Signoretti et al., J. Nat. Cancer Inst., Vol. 92, No. 23, p. 1918 (December 2000) and Gu et al., Oncogene, 19, 1288-1296 (2000). Grades of 1+ to 3+ represent increased intensity of staining with 3+ being strong, dark brown staining Scoring criteria was also based on total percentage of staining 0=0%, 1=less than 25%, 2=25-50% and 3=greater than 50%. The percent positivity and the intensity of staining for nuclear and cytoplasmic as well as sub-cellular components were analyzed. Both the intensity and percentage positive scores were multiplied to produce one number 0-9. 3+ staining was determined from known expression of the antigen from the positive controls of breast adenocarcinoma.

1. A method of predicting responsiveness of a patient afflicted with metastatic breast cancer to therapy with a VEGF inhibitor comprising:
(a) obtaining a biologic sample from the patient; and
(b) determining the expression level of at least two, at least four or at least six biomarkers in said biologic sample, wherein the biomarkers are selected from the group consisting of VEGF, S100A3, PIGO, COL6A1, PSG1, F2RL1, MMP2, KIAA1539, MAP4K2, ITGB4 and CAPN1.

2. The method of claim 1 wherein the biologic sample obtained is selected from the group consisting of tissue, blood, peripheral blood mononuclear cells (PBMC), isolated blood cells, serum and plasma.

3. The method of claim 1 wherein the expression level determined is of the biomarker protein by immunohistochemical (IHC) methods.

4. The method of claim 3 wherein the IHC method is an immunoassay or array.

5. A method of predicting responsiveness of a patient afflicted with metastatic breast cancer to therapy with a VEGF inhibitor comprising:
(a) obtaining a biologic sample from the patient; and
(b) determining the expression level of at least two, at least four or at least six biomarkers in said biologic sample, wherein the biomarkers are selected from the group consisting of VEGF, S100A3, PIGO, COL6A1, PSG1 and F2RL1; or VEGF, MMP2, KIAA1539, MAP4K2, ITGB4 and CAPN1.

6. The method of claim 5 wherein the biologic sample obtained is selected from the group consisting of tissue, blood, peripheral blood mononuclear cells (PBMC), isolated blood cells, serum and plasma.

7. The method of claim 5 wherein the expression level determined is of the biomarker protein by immunohistochemical (IHC) methods.

8. The method of claim 7 wherein the IHC method is an immunoassay or array.

9. The method of claim 5 wherein the expression level of at least two, at least four or at least six biomarkers is determined.

10. A kit comprising an agent for detecting the presence or level in a biologic sample of each of at least two, at least four or at least six biomarkers selected from the group consisting of VEGF, S100A3, PIGO, COL6A1, PSG1, F2RL1, MMP2, KIAA1539, MAP4K2, ITGB4 and CAPN1.

11. The kit of claim 10, wherein the agent is an antibody or a fragment thereof.

12. An array comprising at least one polynucleotide probe complementary and hybridizable to each of an expression product of at least one gene, said gene selected from the group consisting of VEGF, S100A3, PIGO, COL6A1, PSG1, F2RL1, MMP2, KIAA1539, MAP4K2, ITGB4, CAPN1 and combinations thereof.

13. A method of determining the likelihood of that a patient afflicted with metastatic breast cancer will respond to therapy with a VEGF inhibitor, comprising:
(a) determining the level of at least two, at least four or at least six gene transcripts in a biologic sample obtained from said patient corresponding to the biomarkers selected from the group consisting of VEGF, S100A3, PIGO, COL6A1, PSG1, F2RL1, MMP2, KIAA1539, MAP4K2, ITGB4 and CAPN1;
(b) comparing each of the levels determined according to step (a) with the level of each of the same gene transcripts in a biologic sample from a person not afflicted with metastatic breast cancer or a standard level; and
(c) determining whether a correlation exists between the data compared in step (b), wherein said correlation is indicative of said patient having a likelihood of responding the therapy with a VEGF inhibitor.

14. A method of determining the likelihood of that a patient afflicted with metastatic breast cancer will respond to therapy with a VEGF inhibitor, comprising:
(a) obtaining a sample from the patient;
(b) contacting the sample with at least two, at least four or at least six antibodies, wherein said at least two, at least four or at least six antibodies individually bind to a separate biomarker, said biomarker being selected from the group consisting of VEGF, S100A3, PIGO, COL6A1, PSG1, F2RL1, MMP2, KIAA1539, MAP4K2, ITGB4 and CAPN1; and
(b) assessing the patient's likely prognosis based upon a pattern of binding or lack of binding of the at least two, at least four or at least six antibodies to the sample, wherein a pattern of increased or higher level of binding correlates with a higher likelihood that a patient will respond to therapy with the VEGF inhibitor.

15. The method of claim 14, wherein responsiveness to therapy is measured by a reduction in tumor size or burden.

16. The method of claim 15, wherein tumor size is decreased by at least 30%.
